(19) United States
(12) Patent Application Publication
(10) Pub. No.: US 2014/0188092 A1
(43) Pub. Date: Jul. 3, 2014

(54) FOCUSED NEAR-INFRARED LASERS FOR NON-INVASIVE VASECTOMY AND OTHER THERMAL COAGULATION OR OCCLUSION PROCEDURES

(71) Applicant: OMNI MEDSCI, INC., Ann Arbor, MI (US)

(72) Inventor: Mohammed N. Islam, Ann Arbor, MI (US)

(73) Assignee: OMNI MEDSCI, INC., Ann Arbor, MI (US)

(21) Appl. No.: 14/108,995

(22) Filed: Dec. 17, 2013

Related U.S. Application Data
(60) Provisional application No. 61/747,481, filed on Dec. 31, 2012.

(51) Int. Cl. A61B 18/22

(52) U.S. Cl. CPC A61B 18/22
USPC 606/3; 606/15

(57) ABSTRACT

Focused infrared light at wavelengths selected to target tissue below the skin may be used in a non-invasive procedure for vasectomies, varicose veins, hemorrhoids, or fungal nail infections. Infrared light from various sources selected for a particular application may be focused so that the cone of light has lower intensity on the skin/outer tissue and higher intensity at a desired depth to cause thermal coagulation or occlusion of the target tissue beneath the skin. Surface cooling techniques, such as cryogenic sprays or contact cooling may be used to protect the skin. More generally, the focused infrared light with or without surface cooling may be used in applications for thermally coagulating or occluding relatively shallow vessels while protecting or minimizing damage to outer layers of the tissue or skin.

### CROSS-REFERENCE TO RELATED APPLICATIONS

This application claims the benefit of U.S. provisional application Ser. No. 61/747,481 filed Dec. 31, 2012, the disclosure of which is hereby incorporated in its entirety by reference herein.

This application is related to U.S. provisional application Ser. Nos. 61/747,477 filed Dec. 31, 2012; Ser. No. 61/747,472 filed Dec. 31, 2012; Ser. No. 61/747,485 filed Dec. 31, 2012; Ser. No. 61/747,487 filed Dec. 31, 2012; Ser. No. 61/747,492 filed Dec. 31, 2012; Ser. No. 61/747,553 filed Dec. 31, 2012; and Ser. No. 61/754,698 filed Jan. 21, 2013, the disclosures of which are hereby incorporated in their entirety by reference herein.

This application is being filed concurrently with International Application No. ______ entitled Near-Infrared Lasers For Non-Invasive Monitoring Of Glucose, Ketones, HBA1C, And Other Blood Constituents (Attorney Docket No. OMNI0101PCT); International Application ______ entitled Short-Wave Infrared Super-Continuum Lasers For Early Detection Of Dental Caries (Attorney Docket No. OMNI0102PCT); International Application ______ entitled Short-Wave Infrared Super-Continuum Lasers For Natural Gas Leak Detection, Exploration, And Other Active Remote Sensing Applications (Attorney Docket No. OMNI0104PCT); U.S. Application ______ entitled Short-Wave Infrared Super-Continuum Lasers For Detecting Counterfeit Or Illicit Drugs And Pharmaceutical Process Control (Attorney Docket No. OMNI0105PUSP); U.S. Application ______ entitled Non-Invasive Treatment Of Varicose Veins (Attorney Docket No. OMNI0106PUSP); and U.S. Application ______ entitled Near-Infrared Super-Continuum Lasers For Early Detection Of Breast And Other Cancers (Attorney Docket No. OMNI0107PUSP), the disclosures of which are hereby incorporated in their entirety by reference herein.

### TECHNICAL FIELD

This disclosure relates to lasers and light sources for healthcare, medical, or bio-technology applications including systems and methods for using focused near-infrared light sources for non-invasive vasectomy and other thermal coagulation or occlusion procedures.

### BACKGROUND AND SUMMARY

Vasectomy is a relatively simple procedure that causes male sterilization and/or permanent birth control. Men generally have little side effects from vasectomy, and there should also not be any change in sexual performance or function. Also, the vasectomy usually has a higher success rate, lower morbidity and mortality rate, is less expensive, and is easier to perform than female sterilization (tubal ligation). However, despite these advantages, female sterilization is more commonly performed. In the US, for example, in 2009 there were approximately 500,000 vasectomies and 1 million tubal ligations performed. Male fears of complications are frequently cited as the hesitancy for performing vasectomies. Worldwide, approximately 40 million men have had a vasectomy. Complication rates of vasectomy range from 1-6%, and these are often related to lack of experience of the physician performing the procedure. A non-invasive method of performing vasectomies may eliminate the risks of infection, bleeding and scrotal pain as well as reduce the fear associated with surgery, and thus lead to a greater male acceptance of vasectomy.

In a vasectomy surgical procedure, the vas deferens is severed and then tied and/or sealed in a manner to prevent exit of sperm. Typically, a needle is used to inject local anesthesia around the vas, producing a vasal nerve block. Then, approximately centimeter long incisions are made through the vas scrotal skin until the vas is exposed. A segment of the vas is then removed and ends of the vas are occluded using thermal cautery, followed by the placement of hemoclips. In comparison, an incision-less and puncture-less method of performing vasectomies would eliminate the need for surgery and the associated risks.

One option developed recently is a “no-scalpel” vasectomy technique to minimize complications associated with incision during the procedure. However, these techniques still require a puncture through the skin and do not completely eliminate the possibility of bleeding, infection, and scrotal pain. Another alternative is a percutaneous approach to vasectomy using chemical ablation with cyanoacrylate and phenol. For example, a needle may be placed into the lumen of the vas and tests may be run involving dye injections for confirmation. However, this technique may require a high level of skill, since percutaneous access is required to the approximately 300 micron diameter lumen of the vas deferens.

In yet another approach, the use of ultrasound as a non-invasive technique for vas occlusion has been studied. The ultrasound generally requires a coupling medium, which may obstruct the urologist's field-of-view. Also, focused ultrasound may create lesions with a higher depth-to-width ratio, which may damage tissue structures immediately surrounding the vas. In an alternate approach, thermal methods of vas occlusion have also been studied for producing more reliable vas occlusion. For example, it is common for physicians to cauterize the cut ends of the vas. There is also some evidence that a more uniform thermal necrosis of the vas lumen with a hot wire rather than a superficial lumen destruction using electro-cautery provides more successful results.

As described in this disclosure, in one embodiment a non-invasive vasectomy method may use focused infrared light and, possibly, surface cooling. The near infrared wavelength range may provide sufficient penetration depth to pass through the scrotum skin and vas wall, and the particular wavelengths of light may be selected to coagulate or occlude through thermal heating of water in the vas lumen. Several locations on the vas deferens may be coagulated thermally to increase the probability of success. A clamp may be used to secure the vas deferens and scrotum skin. The laser light may be brought in proximity to the patient using a light guide or fiber optics, and a lens and/or mirror system may be used to focus the light near the clamp end. Then, the scrotum skin may be spared of damage by using surface cooling and/or focused light. Surface cooling methods may be borrowed from dermatology, such as a cryogenic cooling spray or a liquid-cooled surface that may be transparent to the light. In addition, by using focused light, the intensity of the light in the scrotum skin may be lower than in the vas lumen. Also, the light may be modulated to control the thermal diffusion into adjacent regions. Using this technique the vas deferens may be coagulated without damaging or puncturing the scrotum skin layer. Thus, focused infrared vasectomy may be a rapid, cost-effective, out-patient procedure with minimal collateral damage and shorter recovery time.

In one embodiment, a therapeutic system includes a light source generating an output optical beam comprising a plurality of semiconductor sources generating an input optical beam, a multiplexer configured to receive at least a portion of the input optical beam and to form an intermediate optical beam, and one or more fibers configured to receive at least a portion of the intermediate optical beam and to form the output optical beam, wherein the output optical beam comprises one or more optical wavelengths, and wherein at least a portion of the one of more fibers is a fused silica fiber with a core diameter less than approximately 400 microns. An interface device is configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample. At least the part of the delivered portion of the output optical beam penetrates into the sample a depth of 1.5 millimeters or more, and at least some of the part of the delivered portion of the output optical beam is at least partially absorbed in the sample to thermally damage at least a part of the sample. The output optical beam comprises a fluence less than about 250 Joules per centimeter squared.

In another embodiment, a therapeutic system includes a light source generating an output optical beam comprising one or more semiconductor sources generating an input optical beam, one or more fibers configured to receive at least a portion of the input optical beam and to form an intermediate optical beam, and a light guide configured to receive at least a portion of the intermediate optical beam and to form the output optical beam, wherein the output optical beam comprises one or more optical wavelengths. An interface device is configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample, and wherein the interface device is a non-invasive device. At least some of the part of the delivered portion of the output optical beam penetrates into the sample a depth of 1.5 millimeters or more, and at least some of the part of the delivered portion of the output optical beam is at least partially absorbed in the sample to thermally damage at least a part of the sample.

In yet another embodiment, a method of therapy includes generating an output optical beam comprising generating an input optical beam from one or more semiconductor sources, forming an intermediate optical beam after propagating at least a portion of the input optical beam through one or more fibers, and guiding at least a portion of the intermediate optical beam and forming the output optical beam, wherein the output optical beam comprises one or more optical wavelengths. The method may also include receiving at least a received portion of the output optical beam and delivering a delivered portion of the output optical beam to a sample, focusing at least a part of the delivered portion of the output optical beam on the sample, and cooling a top surface of the sample. The method may further include absorbing at least some of the part of the delivered portion of the output optical beam in the sample, and damaging thermally at least a part of the sample through a thermal coagulation or occlusion procedure.

### BRIEF DESCRIPTION OF THE DRAWINGS

For a more complete understanding of the present disclosure, and for further features and advantages thereof, reference is now made to the following description taken in conjunction with the accompanying drawings, in which:

FIG. 1 illustrates the typical surgical procedure for a vasectomy. (A) Male Anatomy of a Human. (B) Vas deferens exposed through break in scrotal skin. (C) Vas deferens cut and occluded. (D) Before and after pictures for vasectomy procedure.

FIG. 2 (A) Instruments used in a “no-scalpel” vasectomy technique. (B) Example of the no-scalpel vasectomy surgical approach.

FIG. 3 illustrates a model of the approximate dimensions for the human scrotal skin and vas deferens, particularly when secured within a ring clamp.

FIG. 4 illustrates the overlap of the absorption coefficients for water, adipose, collagen and elastin; vertical lines are also drawn to highlight the wavelengths near 1210 nm and 1720 nm; the adipose and water absorption coefficients are shown on a calibrated scale, while the collagen and elastin are in arbitrary units;

FIG. 5 illustrates the overlap of the absorption coefficients for water and tissue scattering, adipose, collagen and elastin; vertical lines are also drawn to highlight the wavelengths near 1210 nm and 1720 nm; the adipose and water absorption coefficients as well as the scattering loss are shown on a calibrated scale, while the collagen and elastin are in arbitrary units;

FIG. 6 shows the near infrared transmission through porcine muscle tissue, as measured using a Fourier-transform infrared spectrometer.

FIG. 7 illustrates one embodiment of the light input to the non-invasive vasectomy assembly. The light source may comprise, for example, LED's, laser diodes, fiber lasers, or super-continuum lasers.

FIG. 8 shows one embodiment of the non-invasive vasectomy apparatus that may have a focused laser beam and optional cryogenic cooling spray.

FIG. 9 illustrates another embodiment of the non-invasive vasectomy apparatus that may have a focused laser beam and optional surface cooling by flowing fluid in close proximity to the skin.

FIG. 10 shows yet another embodiment of the non-invasive vasectomy apparatus that may comprise multiple collimated or focused light beams and optional surface cooling.

FIG. 11 illustrates that a focusing lens/mirror assembly may be used to create a minimum beam waist near the vas lumen, with lower light intensity in the epidermis and top layer of the dermis.

FIG. 12 shows an experimental set-up for testing chicken breast samples using collimated light. In this experiment, the collimated light has a beam diameter of about 3 mm.

FIG. 13 plots the measured depth of damage (in millimeters) versus the time-averaged incident power (in Watts). Data is presented for laser wavelengths near 980 nm, 1210 nm and 1700 nm, and lines are drawn corresponding to penetration depths of approximately 2 mm, 3 mm, and 4 mm.

FIG. 14 illustrates the optical absorption or density as a function of wavelength between approximately 700 nm and 1300 nm for water, hemoglobin and oxygenated hemoglobin.

FIG. 15 shows a set-up used for in vitro damage experiments using focused infrared light. After a lens system, the tissue is placed between two microscope slides.

FIG. 16 presents histology of renal arteries comprising endothelium, media and adventitia layers and some renal nerves in or below the adventitia. (A) No laser exposure. (B) After focused laser exposure, with the laser light near 1708 nm.

FIG. 17 illustrates the experimental set-up for ex vivo skin laser treatment with surface cooling to protect the epidermis and top layer of the dermis.

FIG. 18 shows MTT histo-chemistry of ex vivo human skin treated with ˜1708 nm laser and cold window (5 seconds precool; 2 mm diameter spot exposure for 3 seconds) at 725 mW (A and B) corresponding to ˜70 J/cm² average fluence and 830 mW (C and D) corresponding to ˜80 J/cm² average fluence.

FIG. 19 illustrates a block diagram or building blocks for constructing high power laser diode assemblies.

FIG. 20 shows a platform architecture for different wavelength ranges for an all-fiber-integrated, high powered, super-continuum light source.

FIG. 21 illustrates one embodiment for a short-wave infrared super-continuum light source.

FIG. 22 shows the output spectrum from the SWIR SC laser of FIG. 21 when ˜10 m length of fiber for SC generation is used. This fiber is a single-mode, non-dispersion shifted fiber that is optimized for operation near 1550 nm.

FIG. 23 illustrates high power SWIR-SC lasers that may generate light between approximately 1.4-1.8 microns (top) or approximately 2-2.5 microns (bottom).

FIG. 24A illustrates a block diagram of one embodiment of an infrared fiber laser operating near 1720 nm;

FIG. 24B shows details of one specific example of an infrared fiber laser operating at approximately 1708 nm; the top part of the figure illustrates one embodiment of the pump fiber laser, and the bottom part of the figure illustrates one embodiment of the cascaded Raman oscillator or cascaded Raman wavelength shifter;

FIG. 25A illustrates a block diagram yet another embodiment of an infrared fiber laser operating near 1210 nm;

FIG. 25B shows details of one specific example of an infrared fiber laser operating at approximately 1212 nm; the top part of the figure illustrates one embodiment of the pump fiber laser, and the bottom part of the figure illustrates one embodiment of the cascaded Raman oscillator or cascaded Raman wavelength shifter.

### DETAILED DESCRIPTION

As required, detailed embodiments of the present disclosure are described herein; however, it is to be understood that the disclosed embodiments are merely exemplary of the disclosure that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

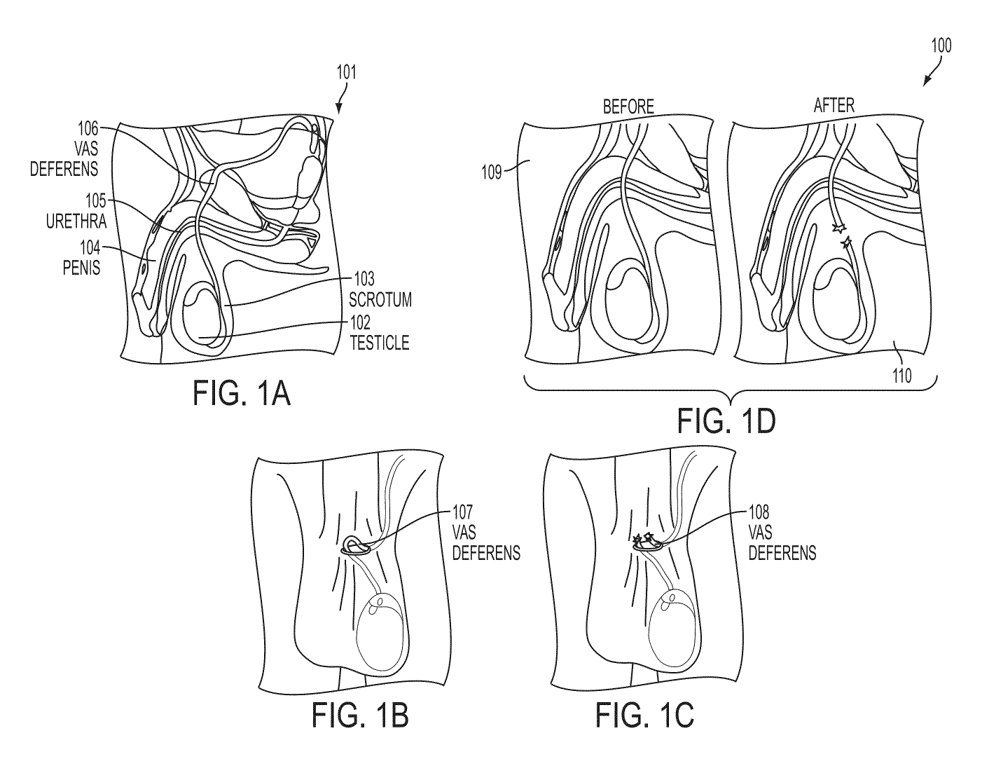

Vasectomies may be performed for male sterilization and/or permanent birth control. The typical surgical procedure 100 for a vasectomy is illustrated in FIG. 1. As an example, FIG. 1A shows the male anatomy of a human 101. Sperm may be produced in the testicles 102, and the sperm is then transported to the urethra 105 in the penis 104 through the vas deferens 106. The scrotum skin 103 may cover the testicles 102 as well as at least part of the vas deferens 106. In a vasectomy surgical procedure, the vas deferens is severed and then tied and/or sealed in a manner to prevent exit of sperm. First, a needle may be used to inject local anesthesia around the vas. Then, approximately centimeter long incisions are made through the scrotal skin 107 until the vas is exposed (FIG. 1B). A segment of the vas may then be removed, and ends of the vas may be occluded 108 using thermal cautery, followed by placement of hemoclips (FIG. 1C). The before 109 and after 110 pictures for the vasectomy procedure are illustrated in FIG. 1D.

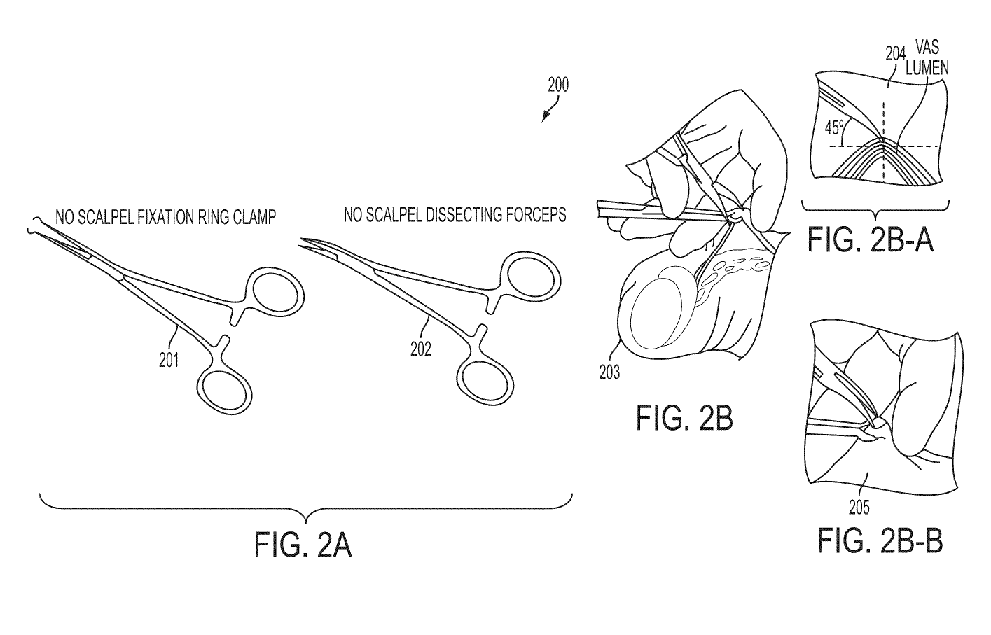

In more recent years, a “no-scalpel” vasectomy technique has been developed that may minimize complications associated with incision during the procedure. Exemplary instruments 200 employed in the no-scalpel vasectomy are illustrated in FIG. 2A. On the left is shown a no-scalpel fixation ring clamp 201, and on the right of FIG. 2A is shown a no-scalpel dissecting forceps 202. In the no-scalpel surgical approach (FIG. 2B), the no-scalpel ring clamp 201 isolates and secures the vas deferens 203 without penetrating the skin. Then, the no-scalpel dissecting forceps 202 pierces the scrotal sac 204 to expose the vas deferens. Next, the vas deferens is lifted out of the scrotum with the no-scalpel dissecting forceps and occluded 205. As FIG. 2 shows, the no-scalpel vasectomy still requires a puncture through the skin and may not completely eliminate the possibility of bleeding, infection and scrotal pain.

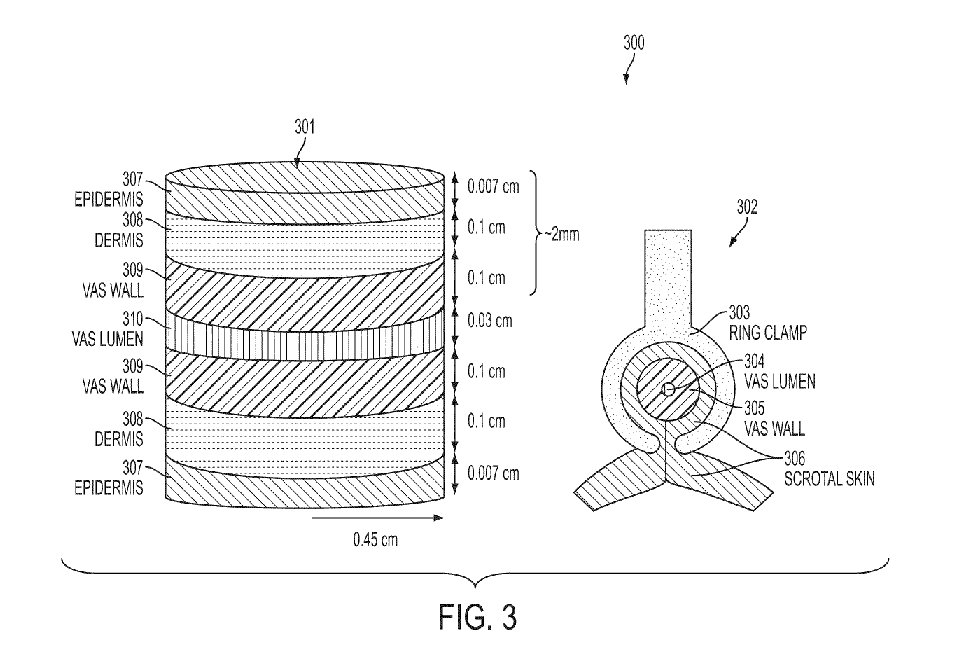

One objective of a non-invasive vasectomy procedure may be to thermally coagulate and scar the vas deferens for permanent occlusion, without the occurrence of adverse side effects such as scrotal skin burns. To accomplish this objective in humans, the dimensions of the scrotal skin and vas deferens should be understood. FIG. 3 illustrates one embodiment of a model of the tissue 300 and the approximate dimensions of the individual tissue layers for human scrotal skin and vas deferens. On the left of FIG. 3 is the cross-section of the tissue 301, and on the right of FIG. 3 the end view of the tissue within the ring clamp 302, such as the no-scalpel fixation ring clamp 201. For example, within the ring clamp 303 is a layer of scrotal skin 306 surrounding the vas wall 305 and approximately in the center the vas lumen 304.

In one embodiment, the cross-sectional dimensions of each of the concentric layers within the ring clamp 303 are illustrated on the left of FIG. 3. The scrotal skin 306 comprises a layer of epidermis 307 (about 70 microns or 0.007 cm) in thickness above about a 1 mm (0.1 cm) thick dermis 308. The vas wall 309 is about 1 mm (0.1 cm) in thickness, and the vas wall 309 may be modeled similar to smooth muscle tissue. The vas lumen 310 is approximately 300 microns in diameter (0.03 cm), and particularly in the infrared the vas lumen 310 may be modeled as water.

In the dermis 308, water may account for approximately 70% of the volume. The next most abundant constituent in the dermis 308 may be collagen, a fibrous protein comprising 70-75% of the dry weight of the dermis 308. Elastin fibers, also a protein, may also be plentiful in the dermis 308, although they constitute a smaller portion of the bulk. In addition, the dermis 308 may contain a variety of structures (e.g., sweat glands, hair follicles with adipose rich sebaceous glands near their roots, and blood vessels) and other cellular constituents.

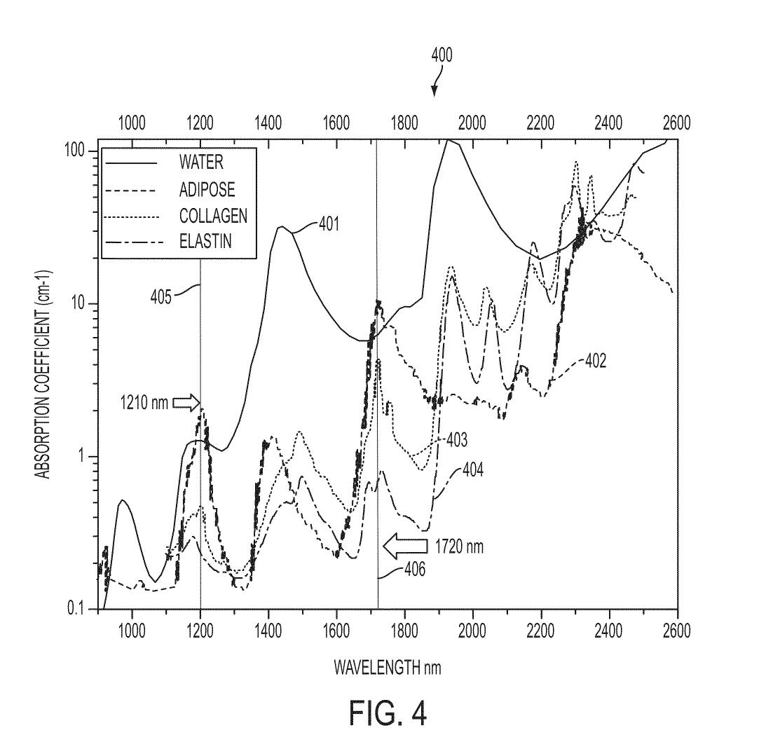

Since in a non-invasive vasectomy technique the light would have to transmit through the dermis 308, the absorption coefficient for the various skin constituents should be examined. For example, FIG. 4 illustrates 400 the absorption coefficients for water (not considering scattering) 401, adipose 402, collagen 403 and elastin 404. Note that the absorption curves for water 401 and adipose 402 are calibrated, whereas the absorption curves for collagen 403 and elastin 404 are in arbitrary units. Also shown are vertical lines demarcating the wavelengths near 1210 nm 405 and 1720 nm 406. In general, the water absorption increases with increasing wavelength. With the increasing absorption beyond about 2000 nm, it may be difficult to achieve deeper penetration into biological tissue in the infrared wavelengths beyond approximately 2500 nm.

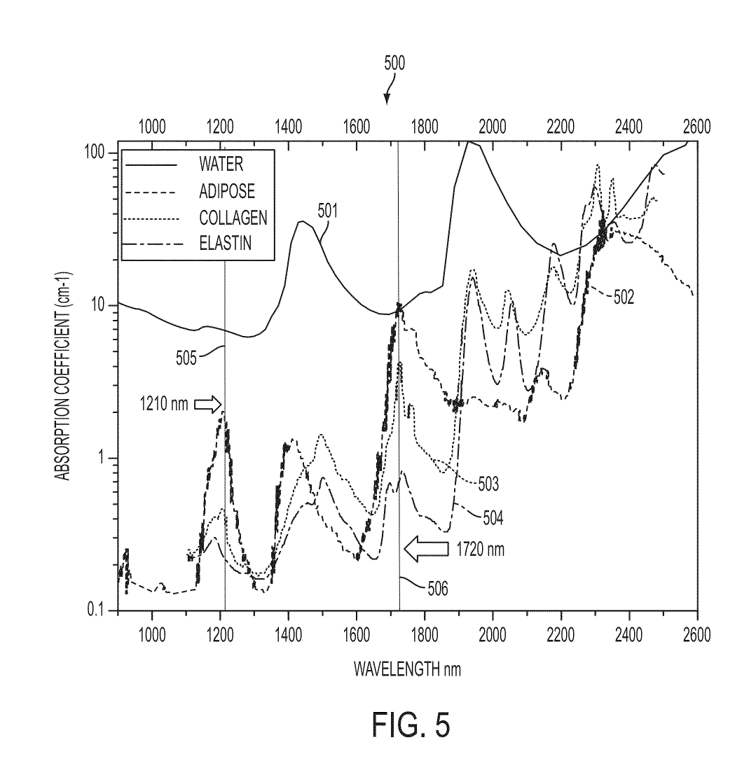

One other consideration may be the scattering through tissue in the dermis. Although the absorption coefficient may be useful for determining the material in which light of a certain infrared wavelength will be absorbed, to determine the penetration depth of the light of a certain wavelength may also require the addition of scattering loss to the curves. In an exemplary embodiment illustrated in FIG. 5, the water curve 501 includes the scattering loss curve in addition to the water absorption. In particular, the scattering loss can be significantly higher at shorter wavelengths. In one embodiment, near the wavelength of 1720 nm (vertical line 506 shown in FIG. 5), the adipose absorption 502 can still be higher than the water plus scattering loss 501. For tissue that contains adipose, collagen and elastin, such as the dermis of the skin, the total absorption can exceed the light energy lost to water absorption and light scattering near 1720 nm. On the other hand, near 1210 nm the adipose absorption 502 can be considerably lower than the water plus scattering loss 501, particularly since the scattering loss can be dominant at these shorter wavelengths. In FIG. 5 shown are 500 the absorption coefficients for water (with scattering) 501, adipose 502, collagen 503 and elastin 504. Note that the absorption curves for water 501 and adipose 502 are calibrated, whereas the absorption curves for collagen 503 and elastin 504 are in arbitrary units. Also shown are vertical lines demarcating the wavelengths near 1210 nm 505 and 1720 nm 506.

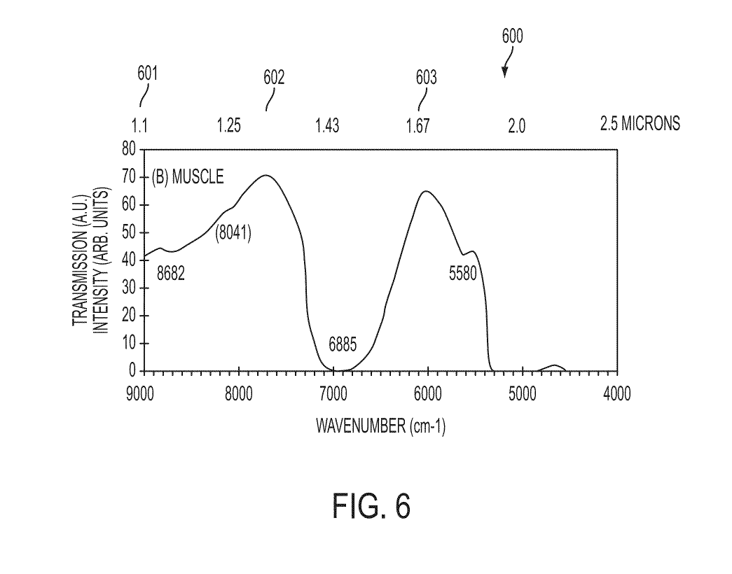

In one embodiment, the vas wall 305, 309 may be modeled as smooth muscle tissue. As an example, smooth muscle tissue or tunica media may comprise protein, which may have an absorption coefficient similar to collagen (e.g., 403 and 503). Hence, by selecting wavelengths near valleys of absorption for collagen 403,503 in FIGS. 4 and 5, the transmission for the light through the vas wall may be higher. In one embodiment, wavelengths below 1100 nm (1.1 microns) or wavelengths near 1310 nm (1.31 microns) may permit transmission through the vas wall as well as reasonable transmission through the dermis. As an example, the near infrared transmission through porcine muscle tissue 600 has been measured using a Fourier-transform infrared spectrometer (FIG. 6). The transmission may be relatively high for wavelengths shorter than about 1100 nm (1.1 microns) 601, near 1310 nm (1.31 microns) 602, and near 1670 nm (1.67 microns) 603. Comparing with FIGS. 4 and 5, these high transmission wavelength ranges correspond approximately to minima in collagen absorption 403,503 as well as areas of relatively low water absorption. In a particular embodiment, light wavelengths near 601, 602 or 603 may be advantageous for minimizing damage to the vas wall.

### Non-Invasive Near Infrared Vasectomy

In one embodiment, one desired goal for a non-invasive vasectomy procedure is to cause coagulation (probably through a thermal means) or occlusion of the vas deferens with minimal damage to the scrotum skin. From FIG. 3, this corresponds to leaving undamaged the top approximately 1 to 1.5 mm or more of skin (epidermis, dermis, and perhaps part of the vas wall). Light can be used to perform the procedure, where the thermal coagulation may occur through heat generated by absorption of the light in the vas lumen and vas wall. One advantage of using light is that the source can be placed remotely, thus not blocking the view of the physician performing the procedure. Also, the optical technique may be non-contact, with no need for a coupling medium (such as generally required in ultrasound). Moreover, the approximately circular optical beam may create circular lesions that may better match the geometry of the vas wall and lumen. In addition, several spots or lengths of the vas deferens may be coagulated or occluded, thereby increasing the probability of success of the procedure.

For a light-based vasectomy, the wavelength of light may be selected to achieve a non-invasive procedure. First, the light should be able to penetrate deep enough to reach through the scrotum skin and vas wall to the vas lumen—e.g., a depth of penetration of approximately 1.5 mm to 2 mm or more. For example, the penetration depth may be defined as the inverse of the absorption coefficient, although it may also be necessary to include the scattering for the calculation. More generally, the light penetration should be deep enough to permit adequate light intensity in the vas lumen to cause thermal coagulation or occlusion. Second, to generate the heat for coagulation, the light should be at least partially absorbed in the vas lumen (which may be modeled as water) and perhaps also at least the interior side of the vas wall (tissue also has a significant water content).

A light based procedure may also be aided by several strategies for preserving the top layers of the scrotum skin. In one embodiment, the light could be focused to a depth of approximately that of the vas lumens. By focusing the light, a funnel may be created for the light intensity, with a lower intensity on the epidermis and dermis layers and higher intensity in the vas lumen. In another embodiment, surface cooling may be added to preserve the epidermis and at least a fraction of the dermis. For example, surface cooling may be a common technique used in laser based dermatology and cosmetic surgery applications. Surface cooling methods may include a cryo-spray, air cooling, or a water/liquid cooled surface in contact with the skin. The water/liquid cooled surface may be in contact surrounding the laser beam spot, or the laser beam may transmit through the surface if it is at least partially transmitting at the laser wavelength. Although two techniques for preserving the scrotum have been described, combinations of the two or other techniques may also be used and are intended to be covered by this disclosure.

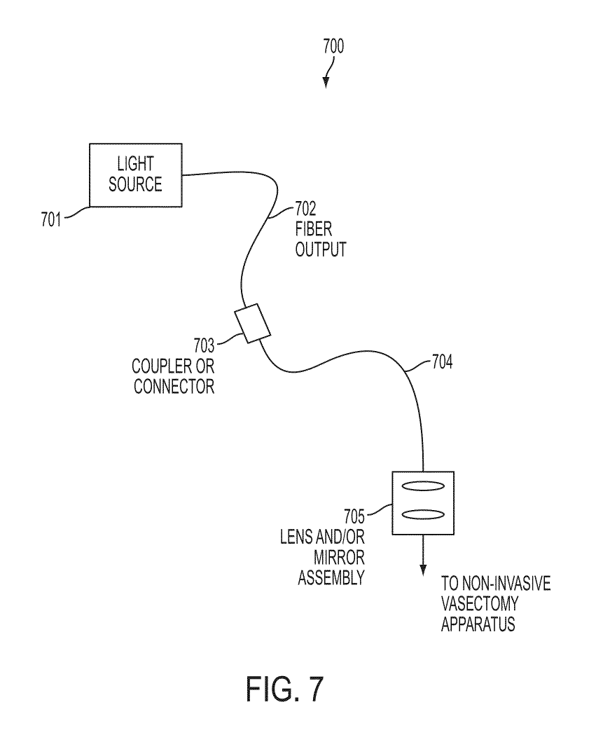

In one embodiment, the light input 700 to the non-invasive vasectomy assembly may be as shown in FIG. 7. The light source 701 may be one or more laser diodes, a fiber laser, or perhaps even a lamp or LED's (various exemplary light sources are described herein). The light source output may be delivered through a light pipe 702, which may be one or more single mode or multi-mode fibers. In a particular embodiment, the light source output pipe 702 may be attached to a coupler or connector 703. In turn, the light pipe or fiber optic or fiber bundle 704 may be coupled to the connector 703, and the light may then be delivered to a lens and/or mirror assembly 705 coupled to the non-invasive vasectomy apparatus. Although one example is shown in FIG. 7, various components may be added or removed from the light source assembly 700, and these variations are intended to be covered by this disclosure.

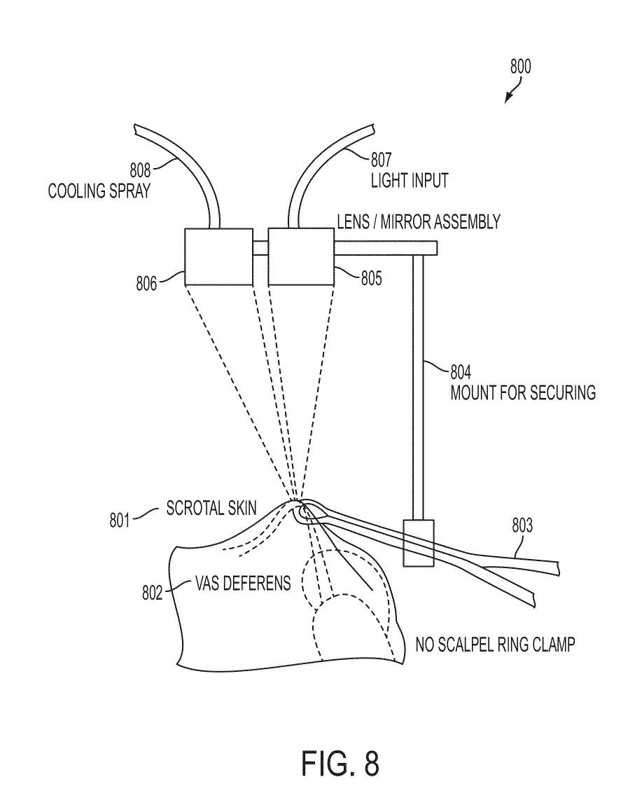

One embodiment of the non-invasive vasectomy apparatus 800 is illustrated in FIG. 8. In particular, this embodiment contemplates a focused laser beam assembly 805 with an optional cryogenic cooling spray attachment 806. The scrotal skin 801 and vas deferens 802 may be held using, for example, a no-scalpel ring clamp 803, such as 201 shown in FIG. 2. Then, a mount or assembly 804 may be used to secure the light input and cooling spray input relative to the ring clamp holding position. Attached to the mount 804 may be a lens and/or mirror assembly 805 that may receive the light input 807 from an apparatus such as in FIG. 7 and then collimate or focus the light onto the scrotal skin 801. The cooling spray head 806 may also be attached to the mount 804, and the head 806 may receive a cooling spray 808 from an external unit. As one particular embodiment, the cooling spray 806 and/or 808 may be a dynamic cooling device made by Candela Laser. The end of the ring clamp 803 may be made out of a material that is transparent to the laser light, or the laser light may hit a spot in close proximity to where the ring clamp is holding the scrotal skin 801. The spray 806 may cool the area near and surrounding where the laser beam is incident on the scrotal skin 801. Although one embodiment is shown in FIG. 8, some of the parts may be removed or other parts may be added, and these variations are also intended to be covered by this disclosure.

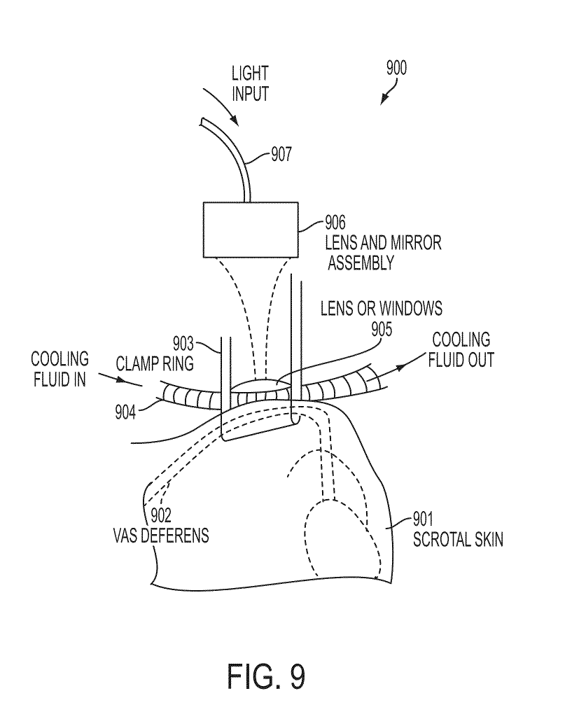

Another embodiment of the non-invasive vasectomy apparatus 900 is illustrated in FIG. 9. In this embodiment, optionally a surface cooling apparatus 904 may be used, where a cooling fluid may be flowed either touching or in close proximity to the scrotal skin 901. In this particular embodiment, a clamp ring is shown that may hold a cylindrical length of the scrotal skin 901 and vas deferens 902, where the cylindrical length may be several millimeters in length. The clamp ring 903 may use two non-scalpel ring clamps 201 on each side, or it may be a modified ring clamp with a cylindrical tip. In this example, a window 905 is also shown on the cylindrical surface for permitting the light to be incident on the scrotal skin 901 and vas deferens 902, and the window 905 may also be a lens. For instance, if a round spot is desired, then a circular or spherical lens window 905 may be used. On the other hand, if a line is desired, then a cylindrical lens window 905 may be used. One advantage of placing a lens 905 in close proximity to the scrotal skin 901 and vas deferens 902 may be that a high numerical aperture, NA, lens may be used, so the cone angle of the light can be relatively steep. A high NA lens may help to increase the difference in light intensity between the scrotal skin 901 and the vas deferens 902. The light input 907 may be received from a light source as shown in FIG. 7. A lens and/or mirror assembly 906 may be used to couple the light input 907 to the lens or window 905, either directly or indirectly. The lens and/or mirror assembly 906 may also be coupled to the clamp ring assembly 903. Although one embodiment is shown in FIG. 9, some of the parts may be removed or other parts may be added, and these variations are also intended to be covered by this disclosure.

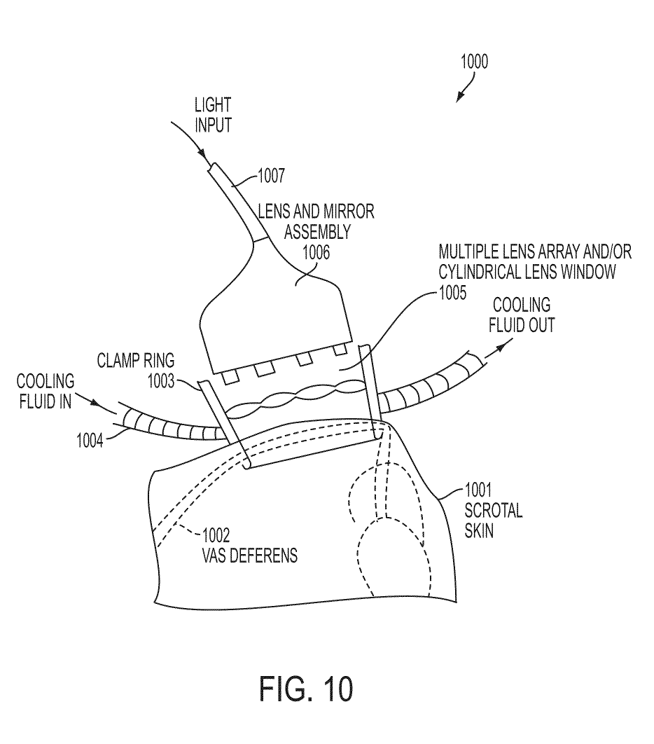

In some instances it may be desirable to create multiple locations of focused light on the vas deferens. For example, the reliability or completeness of the vasectomy may be increased by causing thermal coagulation or occlusion at multiple locations One way to accomplish this may be to slide the assemblies and/or the light source such as shown in FIG. 8 or 9 along the length of the vas deferens. In yet another embodiment shown in FIG. 10, multiple collimated or focused light beams may be created in one assembly 1000. In this embodiment, optionally a surface cooling apparatus 1004 may be used, where a cooling fluid may be flowed either touching or in close proximity to the scrotal skin 1001. Also, in this particular embodiment a clamp ring is shown that may hold a cylindrical length of the scrotal skin 1001 and vas deferens 1002, where the cylindrical length may be several millimeters in length. The clamp ring 1003 may use two non-scalpel ring clamps 201 on each side, or it may be modified ring clamp with a cylindrical tip. The light input 1007 may be received from a light source as shown in FIG. 7, which may use a fiber or fiber bundles to couple the light to the lens/mirror assembly 1006. A lens and/or mirror assembly 1006 may be used to couple the light input 1007 to the lenslet array or window 1005, either directly or indirectly. The lens and/or mirror assembly 1006 may also be coupled to the clamp ring assembly 1003.

In the embodiment of FIG. 10, a window and/or lenslet array 1005 is also shown on the cylindrical surface for permitting the light to be incident on the scrotal skin 1001 and vas deferens 1002 at multiple spots. The lenslet array 1005 may comprise circular, spherical or cylindrical lenses, depending on the type of spots desired. As before, one advantage of placing the lenslet array 1005 in close proximity to the scrotal skin 1001 and vas deferens 1002 may be that a high NA lens may be used. Also, the input from the lens and/or mirror assembly to the lenslet array 1005 may be single large beam, or a plurality of smaller beams. In one embodiment, a plurality of spots may be created by the lenslet array 1005 to cause a plurality of locations of thermal coagulation in the vas lumen along the vas deferens 1002. Although four spots are shown in FIG. 10, any number of spots may be used and are intended to be covered by this disclosure. One advantage of having the plurality of spots in close proximity to each other over a distance along the vas deferens of several millimeters or even a centimeter or more may be that a vasectomy reversal may be permissible. For instance, if a reversal of the vasectomy is desired, then micro-surgery may be conducted to cut out the region of thermal coagulation, and then the two ends of the vas deferens 1005 can then be rejoined.

Although several embodiments of non-invasive vasectomy apparatuses are illustrated in FIGS. 8-10, some of the parts may be removed or other parts may be added, and these variations are also intended to be covered by this disclosure. Also, different combinations of these techniques may be employed, and other techniques may also be used and are intended to be covered by this disclosure. For example, in some instances only focused light may be used, in other instances only surface cooling or cryogenic sprays may be used, and in yet other embodiments a combination of the two may be used. Moreover, the clamp ring may comprise one or more clamps, and a cylindrical end may be attached to or separate from the ring clamps. Some or all of the ring clamps may be transparent to the light, or the light may be focused to a region in close proximity to the ring clamps. These and other variations are also intended to be covered by this disclosure.

### Focusing and/or Surface Cooling

One goal of this disclosure is to provide a method of causing coagulation or occlusion of sections of the vas deferens with minimal damage to the scrotum skin. One method of achieving this goal may be to focus the light, so that low intensity may be incident on the scrotum skin, while higher intensity of light may be incident on the vas deferens wall and lumen. Another method of achieving this goal may be to add surface cooling of the epidermis and dermis, such as using cryogenic spray or liquid-cooled surface contact—techniques that are commonly used in dermatology and cosmetic surgery. In yet another method, some combination of light focusing and surface cooling may be employed. These are provided as particular examples, but other methods of minimizing damage to the scrotal skin may also be used and are intended to be covered by this disclosure.

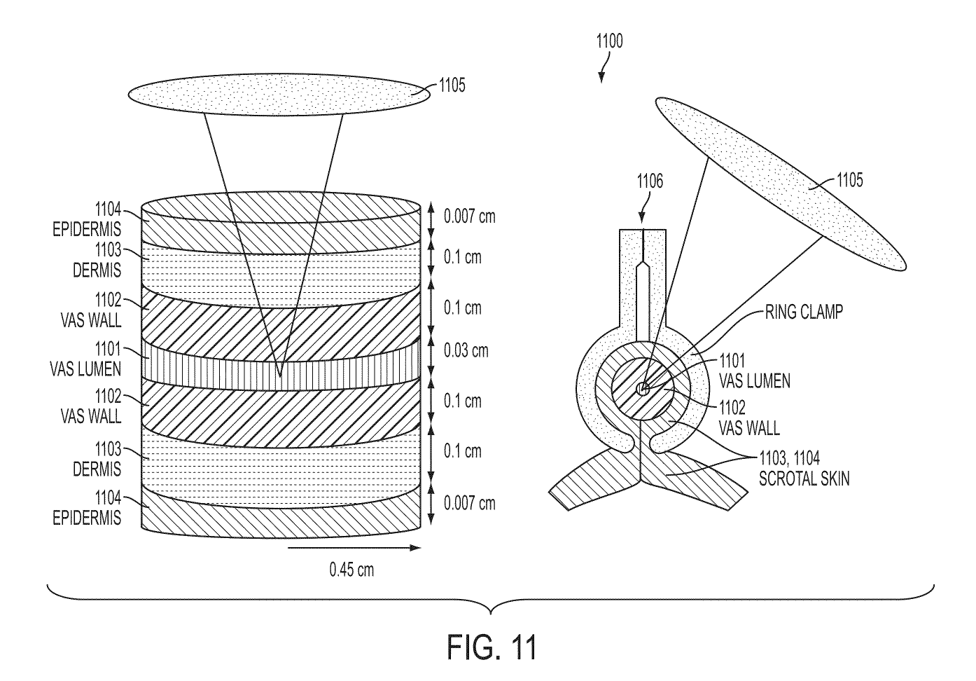

The light to the non-invasive vasectomy assembly, such as in FIGS. 8-10, may be incident from a set-up 700 such as in FIG. 7. The light sources 701 will be discussed in further detail later in this disclosure. In a particular embodiment, the light may be delivered to the scrotal skin and vas deferens using a lens and/or mirror assembly, such as 705, 805, 906, or 1006. A single beam or a plurality of beams may be created and focused or collimated using the lens and/or mirror assembly. In one embodiment 1100 shown in FIG. 11, the light may be focused so the minimum beam waist falls approximately near the vas lumen 1101, thus helping to thermally coagulate the vas lumen in one or a plurality of spots. In one particular embodiment, a plurality of damage spots may be induced, and the damage spots may be in close proximity, thereby permitting reversal of the vasectomy at a later time.

For the focusing arrangement 1100 of FIG. 11, a funnel of light may be implemented, so the intensity of light is lower at the epidermis 1104 and dermis 1103 while higher in the vas wall 1102 and vas lumen 1101. The cone of light may have a beam waist in the vicinity of the vas lumen 1101. The light may be applied adjacent to the ring clamp 1106, through the ring clamp 1106 if it is made of transparent material, or the ring clamp 1106 may itself have a lens or window (e.g., 905, 1105). The lens and/or mirror assembly 1105 may comprise one or more lenses, microscope objectives, curved or flat mirrors, lens tipped fibers, or some combination of these elements. As an example, the optics such as used in a camera may be employed in this arrangement 1105, provided that the optics is transparent at the light wavelengths being used. Moreover, reflections and losses through the optics may be reduced by applying anti-reflection coatings, and chromatic dispersion may be reduced by using reflective optics rather than refractive optics. Although a particular method of focusing the light has been described, other methods may also be used and are intended to be covered by this disclosure.

In a non-limiting example, a plurality of spots may be used, or what might be called a fractionated beam. The fractionated laser beam may be added to the laser delivery assembly or delivery head in a number of ways. In one embodiment, a screen-like spatial filter may be placed in the pathway of the beam to be delivered to the biological tissue. The screen-like spatial filter can have opaque regions to block the light and holes or transparent regions, through which the laser beam may pass to the tissue sample. The ratio of opaque to transparent regions may be varied, depending on the application of the laser. In another embodiment, a lenslet array can be used at or near the output interface where the light emerges. In yet another embodiment, at least a part of the delivery fiber from the infrared laser system to the delivery head may be a bundle of fibers, which may comprise a plurality of fiber cores surrounded by cladding regions. The fiber cores can then correspond to the exposed regions, and the cladding areas can approximate the opaque areas not to be exposed to the laser light. As an example, a bundle of fibers may be excited by at least a part of the laser system output, and then the fiber bundle can be fused together and perhaps pulled down to a desired diameter to expose to the tissue sample near the delivery head. In yet another embodiment, a photonic crystal fiber may be used to create the fractionated laser beam. In one non-limiting example, the photonic crystal fiber can be coupled to at least a part of the laser system output at one end, and the other end can be coupled to the delivery head. In a further example, the fractionated laser beam may be generated by a heavily multi-mode fiber, where the speckle pattern at the output may create the high intensity and low intensity spatial pattern at the output. Although several exemplary techniques are provided for creating a fractionated laser beam, other techniques that can be compatible with optical fibers are also intended to be included by this disclosure.

In a further embodiment, it may be advantageous to apply surface cooling techniques to minimize damage to the epidermis 1104 and dermis 1103. In a particular embodiment, the surface cooling may be accomplished by having a thermally conductive surface approximately in contact with the scrotal skin, as illustrated 904 in FIG. 9 or 1004 in FIG. 10. Liquid coolant may flow in proximity to the skin, helping thereby thermally conducting away some of the heat. The cooling fluid may be water, Freon, or other liquids that may have a lower freezing temperature.

In yet another embodiment, the surface cooling may be accomplished using a dynamic cooling device, such as a cryogenic spray. As an example, FIG. 8 illustrates a cooling spray 808 that may be adjacent to the lens/mirror assembly 805 and mounted on a stand 804. In one particular embodiment, the cooling spray 808 may be a dynamic cooling device made by Candela Laser Corporation. As an example, this device may deliver the cryogen (halocarbon 134a, 1,1,1,2-tetrafluoroethane, boiling point=−26 degree Celsius) to the tissue surface through a solenoid valve. The solenoid valve may be triggered to deliver one or more cryogen pulses to precool the scrotal skin before irradiation using the laser. In addition, the cryogen spray may be delivered continuously or intermittently during or between laser pulses, as well as after the laser irradiation is completed. Moreover, a cryogen mask may be employed to thermally insulate the surrounding scrotal skin from the cryogen spray, thereby avoiding or minimizing superficial freezing burns. Although a particular embodiment is described, other configurations and combinations of focusing and surface cooling may be used and are intended to be covered by this disclosure.

Beyond the use of focused light and surface cooling, other methods may also be used to reduce the potential for pain or damage to the scrotal skin. In yet another embodiment, an optical clearing agent, OCA, may be applied to the scrotal skin to reduce the laser power necessary. The OCA may reduce skin scattering and increase transmission through the skin, thereby reducing the required power levels and the risk of scrotal skin burns. The OCA may also reduce the differences in refractive index between different skin layers and air, thereby reducing the amount of reflected light from refractive index mismatches. Examples of common OCAs include dimethyl sulfoxide, glycerol, glucose and other sugar compounds—as well as mixtures of these compounds. Also, in one embodiment the OCA may be delivered to the skin using a pneumatic jet device, such as a Madajet device made by Advanced Meditech International. For instance, the OCA may be applied near and around the spot(s) of laser irradiation.

In another embodiment, a local anesthetic may be used in the vicinity of the laser irradiation and ring clamp holding. One example of a local anesthesia may be lidocaine. Many local anesthetics may be membrane stabilizing drugs, and local anesthetics may be bases and may usually be formulated as the hydrochloride salt to render them water-soluble. In one embodiment, the Madajet may be used, which is a commercially available device marketed for non-invasive delivery of local anesthesia through the scrotal skin during conventional no-scalpel vasectomy. Beyond optical clearing agents and local anesthesia, other ointments, creams, liquids or sprays may also be applied to the scrotal skin area before, during and after the laser irradiation, and these are also intended to be covered by this disclosure.

Thus, as described above, there are a number of advantages of using focused infrared light for non-invasive vasectomies. First, it can be non-invasive in that sections of the vas deferens can be thermally coagulated or occluded without exposing the vas deferens through the skin. Second, it may be a non-contact method, without the necessity of a coupling medium with the scrotal skin. In turn, the urologists' field-of-view may be preserved, permitting the physicians' monitoring of the progress and noting signs of skin damage. The method may also borrow from a conventional no-scalpel vasectomy approach for separating and isolating the vas deferens under the scrotal skin. Moreover, depending on the optics used, circular or cylindrical lesions may be created that better match the geometry of the vas tube. In addition, several spots along the length of the vas deferens can be coagulated, thereby increasing the probability of success of the procedure. Beyond these, other advantages may also be gained by using focused infrared light in procedures seeking to damage relatively shallow vessels below the skin while minimizing damage to the skin.

### Laser Experiments: Penetration Depth, Focusing, Skin Cooling

Some preliminary experiments show the feasibility of using focused infrared light for non-invasive vasectomy procedures, or other procedures where relatively shallow vessels below the skin are to be thermally coagulated or occluded with minimum damage to the skin upper layers. In one embodiment, the penetration depth and optically induced thermal damage has been studied in chicken breast samples. Chicken breast may be a reasonable optical model for smooth muscle tissue, comprising water, collagen and proteins. Commercially available chicken breast samples were kept in a warm bath (˜32 degree Celsius) for about an hour, and then about half an hour at room temperature in preparation for the measurements.

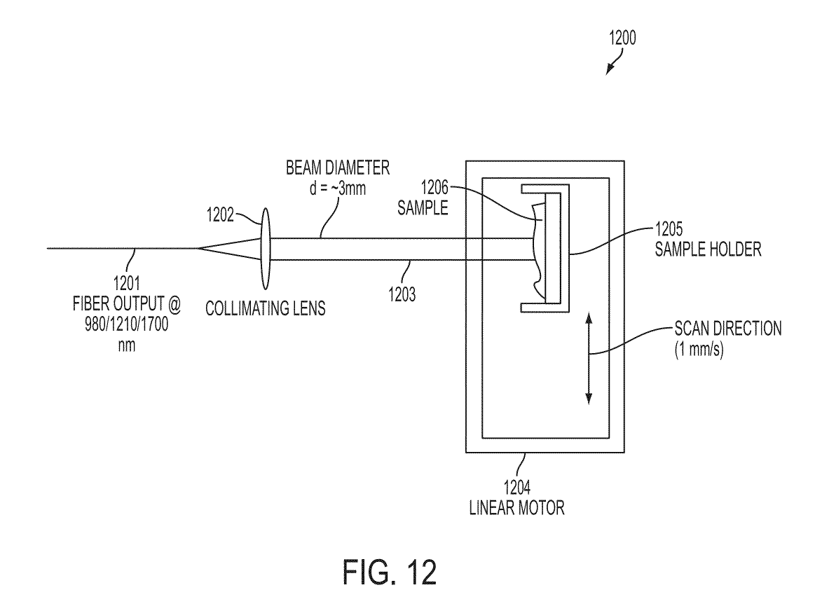

An exemplary set-up 1200 for testing chicken breast samples using collimated light is illustrated in FIG. 12. The laser light 1201 near 980 nm, 1210 nm, or 1700 nm may be provided from one or more laser diodes or fiber lasers, as described further below. In this instance, laser diodes were used, which comprise a plurality of laser diode emitters that are combined using one or more multiplexers (particularly spatial multiplexers), and then the combined beam is coupled into a multi-mode fiber (typically 100 microns to 400 microns in diameter). The output from the laser diode fiber was then collimated using one or more lenses 1202. The resulting beam 1203 was approximately round with a beam diameter of about 3 mm. The beam diameter was verified by blade measurements (i.e., translating a blade across the beam). Also, the time-averaged power was measured in the nearly collimated section after the lens using a large power meter. The chicken breast samples 1206 were mounted in a sample holder 1205, and the sampler holder 1205 was mounted in turn on a translation stage 1204 with a linear motor that could move perpendicular to the incoming laser beam. Although particular details of the experiment are described, other elements may be added or eliminated, and these alternate embodiments are also intended to be covered by this disclosure.

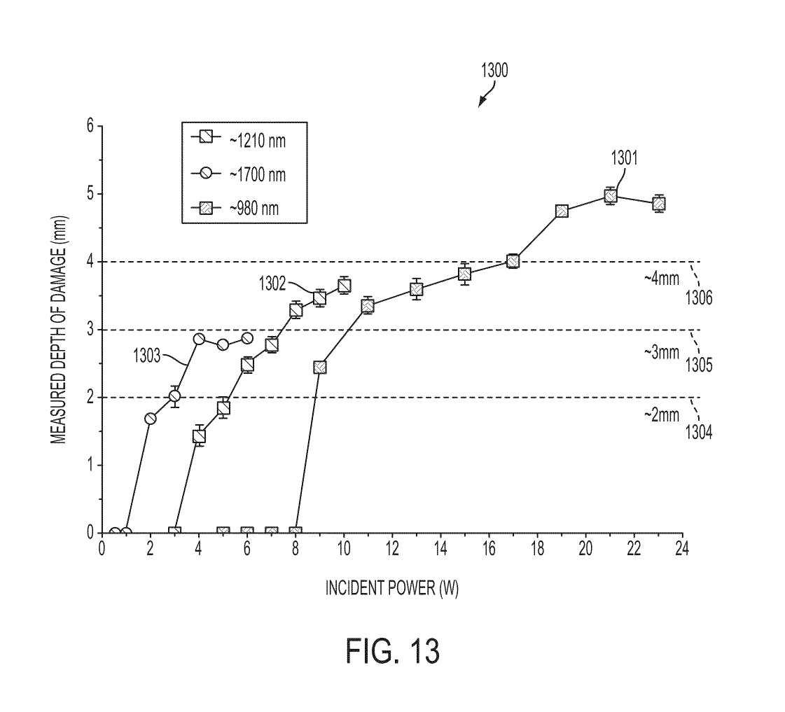

For these particular experiments, the measured depth of damage (in millimeters) versus the incident laser power (in Watts) is shown 1300 in FIG. 13. In this embodiment, laser diodes were used at wavelengths near 980 nm, 1210 nm and 1700 nm. The curve 1301 corresponds to about 980 nm, the curve 1302 corresponds to about 1210 nm, and the curve 1303 corresponds to about 1700 nm. It may be noted that there is a threshold power, above which the damage depth increases relatively rapidly. For example, the threshold power for wavelengths around 980 nm may be about 8 W, the threshold power for wavelengths around 1210 nm may be 3 W, and the threshold power for wavelengths around 1700 nm may be about 1 W. The threshold powers may be different at the different wavelengths because of the difference in water absorption (e.g., 401 in FIG. 4 or 501 in FIG. 5). Part of the difference in threshold powers may also arise from the absorption of proteins such as collagen (e.g., 403 in FIG. 4 or 503 in FIG. 5). After a certain power level, the damage depth appears to saturate: i.e., the slope flattens out as a function of increasing pump power.

In one embodiment, if we define the penetration depth as when the penetration depth begins to approximately saturate, then for wavelengths of about 980 nm 1301 the penetration depth 1306 may be defined as approximately 4 mm, for wavelengths of about 1210 nm 1302 the penetration depth 1305 may be defined as approximately 3 mm, and for wavelengths of about 1700 nm 1303 the penetration depth 1304 may be defined as approximately 2 mm. These are only approximate values, and other values and criteria may be used to define the penetration depth. It may also be noted that the level of damage at the highest power points differs at the different wavelengths. For example, at the highest power point of 1303 near 1700 nm, much more damage is observed, showing evidence of even boiling and cavitation. This may be due to the higher absorption level near 1700 nm (e.g., 401 in FIG. 4). On the other hand, at the highest power point 1301 near 980 nm, the damage is not as catastrophic, but the spot size appears larger. The larger spot size may be due to the increased scattering at the shorter wavelengths (e.g., 501 in FIG. 5). Based on data 1300 such as in FIG. 13, it may be possible to select the particular wavelength for the laser beam to be used in the non-invasive procedure.

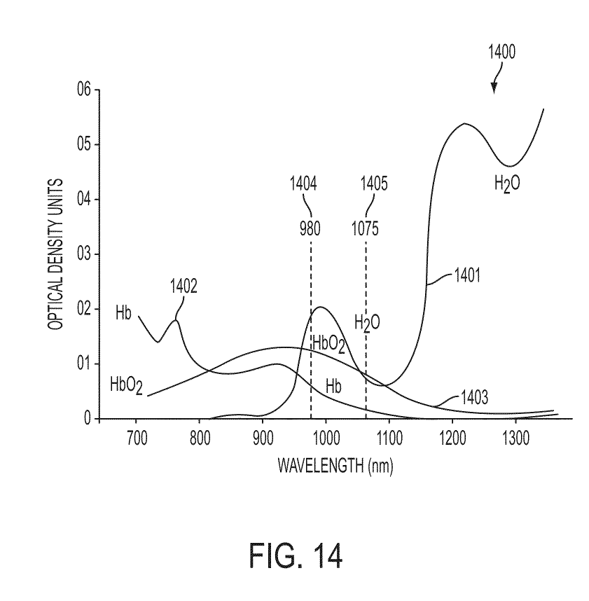

Even near wavelengths such as described in FIG. 13, the particular wavelength selected may be more specifically defined based on the target tissue of interest. In one particular embodiment, the vas lumen may be modeled as water, and for this example assume that wavelengths in the vicinity of 980 nm are being selected to create thermal coagulation or occlusion. FIG. 14 shows the optical absorption or density as a function of wavelength 1400 between approximately 700 nm and 1300 nm. Curves are shown for the water absorption 1401, hemoglobin Hb absorption 1402, and oxygenated hemoglobin HbO₂ 1403. In this example, two particular wavelengths are compared: 980 nm 1404 and 1075 nm 1405. For instance, 980 nm may be generated using one or more laser diodes, while 1075 nm may be generated using an ytterbium-doped fiber laser. If maximizing the penetration depth is the significant problem, then 1075 nm 1405 may be preferred, since it falls near a local minimum in water 1401, hemoglobin 1402, and oxygenated hemoglobin 1403 absorption. On the other hand, if the penetration depth at 980 nm 1404 is adequate and the problem is to generate heat through water absorption, then 980 nm 1404 may be a preferred wavelength for the light source because of the higher water absorption. This wavelength range is only meant to be exemplary, but other wavelength ranges and particular criteria for selecting the wavelength may be used and are intended to be covered by this disclosure.

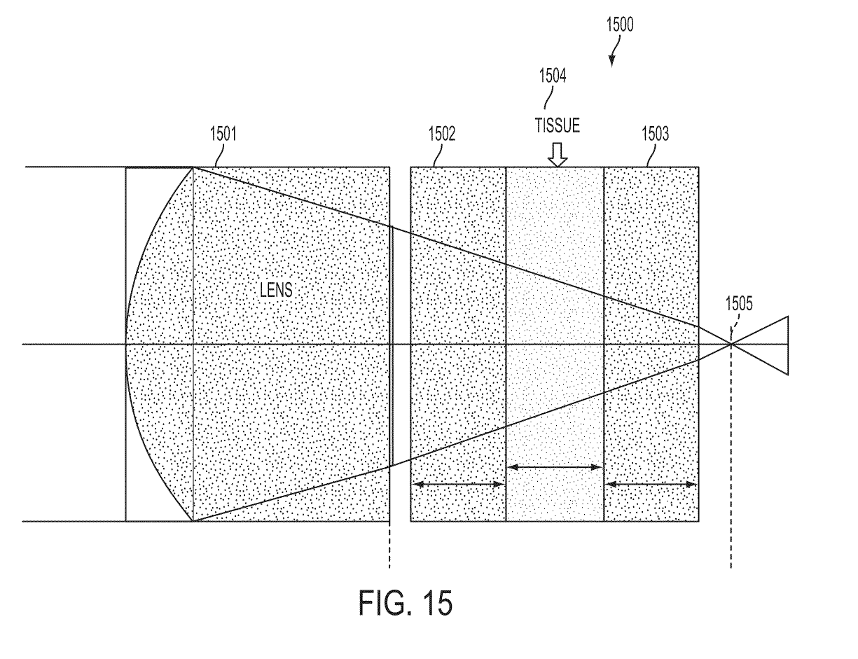

In another embodiment, focused infrared light has been used to preserve the top layer of a tissue while damaging nerves at a deeper level. For instance, FIG. 15 illustrates the set-up 1500 used for the focused infrared experiments. In this embodiment, a lens 1501 is used to focus the light. Although a single lens is shown, either multiple lenses, GRIN (gradient index) lenses, curved mirrors, or a combination of lenses and mirrors may be used. In this particular example, the tissue 1504 is placed between two microscope slides 1502 and 1503 for in vitro experiments. The tissue 1504 is renal artery wall either from porcine or bovine animals (about 1.2 mm thick sample)—i.e., this is the artery leading to the kidneys, and it is the artery where typically renal denervation may be performed to treat hypertension. For this example, the minimum beam waist 1505 falls behind the tissue, and the intensity contrast from the front of the tissue (closest to the lens) to the back of the tissue (furthest from the lens) is about 4:1. These are particular ranges used for this experiment, but other values and locations of minimum beam waist may also be used and intended to be covered by this disclosure.

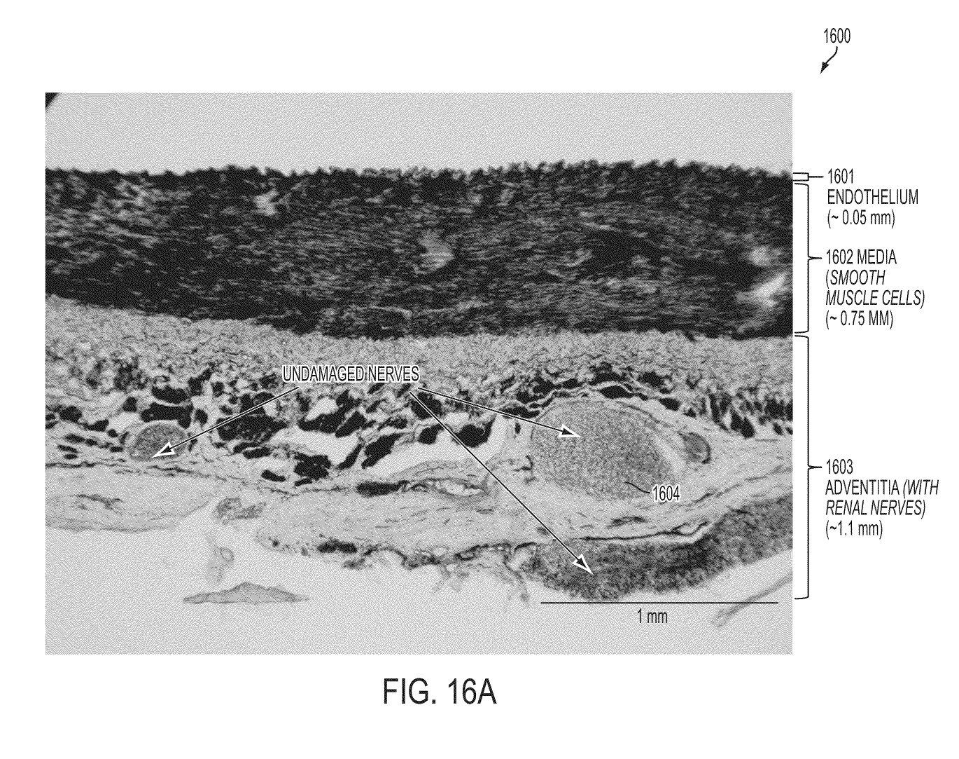

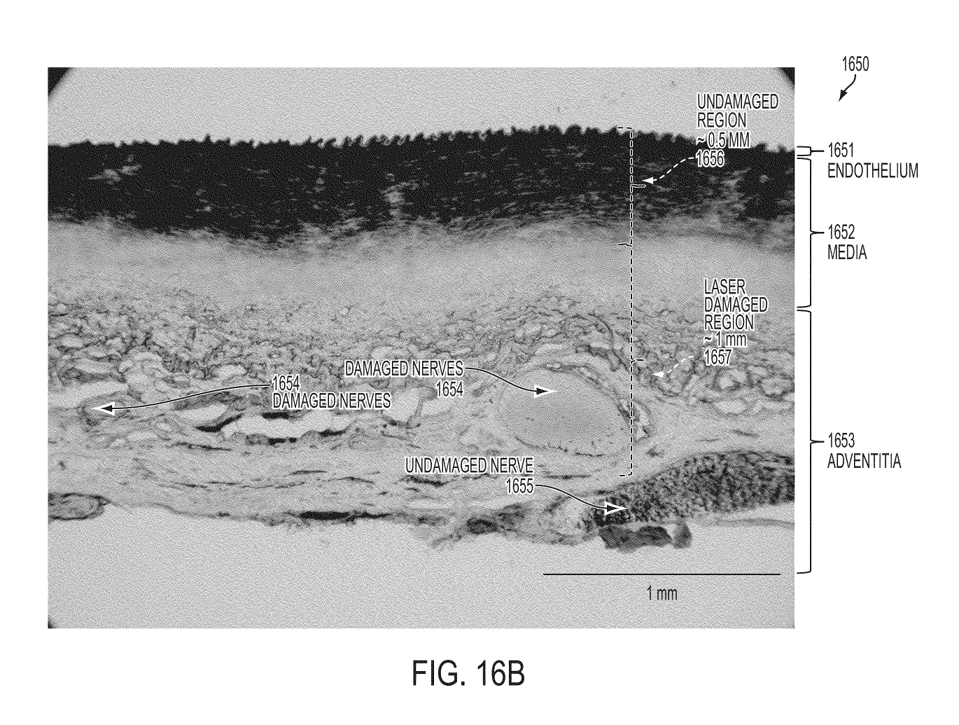

For a particular embodiment, histology of the renal artery is shown in FIG. 16A for no laser exposure 1600 and shown in FIG. 16B with focused infrared laser exposure 1650. In this experiment, the beam diameter incident on the lens was about 4 mm, and the distance from the edge of the flat side of lens to the minimum beam waist was about 3.75 mm. The beam diameter on the front side of the renal artery (i.e., the endothelium side) was about 1.6 mm, and the beam diameter on the back side of the renal artery was about 0.8 mm. In FIG. 16A with no laser exposure, the layers of the artery wall may be identified: top layer of endothelium 1601 that is about 0.05 mm thick, the media comprising smooth muscle cells or tissue 1602 that is about 0.75 mm thick, and the adventitia 1603 comprising some of the renal nerves 1604 that is about 1.1 mm thick. These are particular values for this experiment, and other layers and thicknesses may also be used and are intended to be covered by this disclosure.

The histology with focused infrared light exposure 1650 is illustrated in FIG. 16B. The laser light used is near 1708 nm from a cascaded Raman oscillator (described in greater detail herein), and the power incident on the tissue is about 0.8 W and the beam is scanned across the tissue at a rate of approximately 0.4 mm/sec. The various layers are still observable: the endothelium 1651, the media 1652, and the adventitia 1653. With this type of histology, the non-damaged regions remain darker (similar to FIG. 16A), while the laser induced damaged regions turn lighter in color. In this example, the endothelium 1651 and top layer of the media 1652 remain undamaged—i.e., the top approximately 0.5 mm is the undamaged region 1656. The laser damaged region 1657 extends for about 1 mm, and it includes the bottom layer of the media 1652 and much of the adventitia 1653. The renal nerves 1654 that fall within the damage region 1657 are also damaged (i.e., lighter colored). On the other hand, the renal nerves beyond this depth, such as 1655, may remain undamaged.

Thus, by using focused infrared light near 1708 nm in this example, the top approximately 0.5 mm of the renal artery is spared from laser damage. It should be noted that when the same experiment is conducted with a collimated laser beam, the entire approximately 1.5 mm is damaged (i.e., including regions 1656 and 1657). Therefore, the cone of light with the lower intensity at the top and the higher intensity toward the bottom may, in fact, help preserve the top layer from damage. There should be a Beer's Law attenuation of the light intensity as the light propagates into the tissue. For example, the light intensity should reduce exponentially at a rate determined by the absorption coefficient. In these experiments it appears that the focused light is able to overcome the Beer's law attenuation and still provide contrast in intensity between the front and back surfaces.

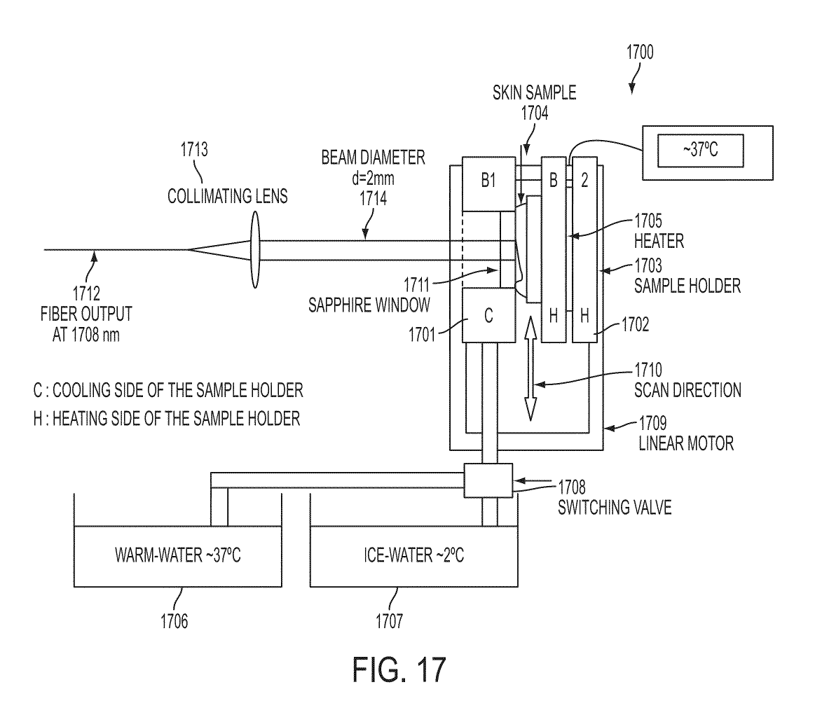

In another embodiment, experiments have also been conducted on dermatology samples with surface cooling, and surface cooling is shown to preserve the top layer of the skin during laser exposure. In this particular example, the experimental set-up 1700 is illustrated in FIG. 17. The skin sample 1704, or more generally sample under test, is placed in a sample holder 1703. The sample holder 1703 has a cooling side 1701 and a heating side 1702. The heating side 1702 comprises a heater 1705, which may be adjusted to operate around 37 degrees Celsius—i.e., close to body temperature. The cooling side 1701 is coupled to an ice-water bath 1707 (around 2 degrees Celsius) and a warm-water bath 1706 (around 37 degrees Celsius) through a switching valve 1708. The entire sample holder 1703 is mounted on a linear motor 1709, so the sample can be moved perpendicular 1710 to the incoming light beam.

In this embodiment, the light is incident on the sample 1704 through a sapphire window 1711. The sapphire material 1711 is selected because it is transparent to the infrared wavelengths, while also being a good thermal conductor. Thus, the top layer of the sample 1704 may be cooled by being approximately in contact with the sapphire window 1711. The laser light 1712 used is near 1708 nm from a cascaded Raman oscillator (described in greater detail herein), and one or more collimating lenses 1713 are used to create a beam with a diameter 1714 of approximately 2 mm. This is one particular embodiment of the sample surface cooling arrangement, but other apparatuses and methods may be used and are intended to be covered by this disclosure.

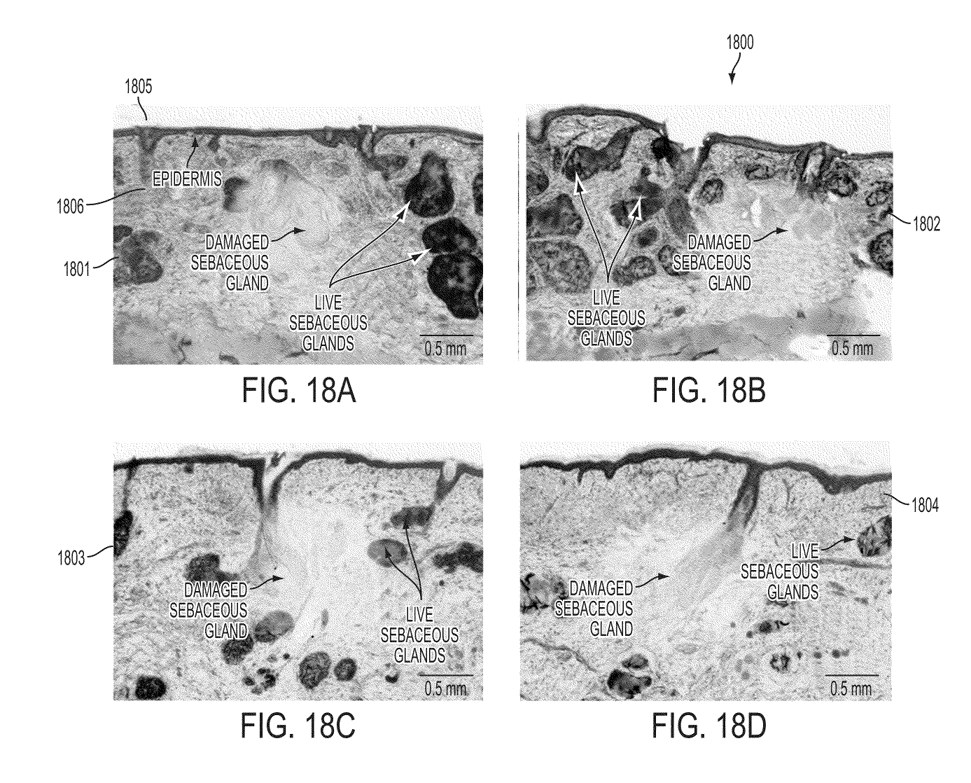

Experimental results obtained using the set-up of FIG. 17 are included in FIG. 18. In this example, FIG. 18 shows the MTT histochemistry of human skin 1800 treated with ˜1708 nm laser (5 seconds pre-cool; 2 mm diameter spot exposure for 3 seconds) at 725 mW (A 1801, B 1802) corresponding to about 70 J/cm² average fluence, and 830 mW (C 1803, D 1804) corresponding to about 80 J/cm² average fluence. The images in FIG. 18 show that the application of a cold window was effective in protecting the epidermis 1805 (darker top layer) and the top approximately 0.4 or 0.5 mm of the dermis 1806. As before, the darker regions of the histology correspond to undamaged regions, while the lighter regions correspond to damaged regions. In contrast, when no surface cooling is applied, then thermal damage to the dermis occurs in the epidermis and dermis where the laser exposure occurs, and the thermal damage extends to about 1.3 or 1.4 mm or more from the skin surface. Thus, surface cooling applied to the skin may help to reduce or eliminate damage to the top layer of the skin under laser exposure.

In summary, experiments verify that infrared light, such as near 980 nm, 1210 nm, or 1700 nm, may achieve penetration depths between approximately 2 mm to 4 mm or more. The top layer of skin or tissue may be spared damage under laser exposure by focusing the light beyond the top layer, applying surface cooling, or some combination of the two. These are particular experimental results, but other wavelengths, methods and apparatuses may be used for achieving the penetration and minimizing damage to the top layer and are intended to be covered by this disclosure. In an alternate embodiment, it may be beneficial to use wavelengths near 1310 nm if the absorption from skin constituents (FIG. 4), such as collagen 403, adipose 402 and elastin 404, are to be minimized. The water absorption 401 near 1310 nm may still permit a penetration depth of approximately 1 cm, or perhaps less. In yet another embodiment, wavelengths near 1210 nm may be beneficial, if penetration depths on the order of 3 mm are adequate and less scattering loss (e.g. 501 in FIG. 5) is desired. Any of FIG. 4, 5, or 13 may be used to select these or other wavelengths to achieve the desired penetration depth and to also perhaps target particular tissue of interest, and these alternate embodiments are also intended to be covered by this disclosure.

### Laser Systems for Therapeutics or Diagnostics

Infrared light sources can be used for diagnostics and therapeutics in a number of medical applications. For example, broadband light sources can advantageously be used for diagnostics, while narrower band light sources can advantageously be used for therapeutics. In one embodiment, selective absorption or damage can be achieved by choosing the laser wavelength to lie approximately at an absorption peak of particular tissue types. Also, by using infrared wavelengths that minimize water absorption peaks and longer wavelengths that have lower tissue scattering, larger penetration depths into the biological tissue can be obtained. In this disclosure, infrared wavelengths include wavelengths in the range of approximately 0.9 microns to 10 microns, with wavelengths between about 0.98 microns and 2.5 microns more suitable for certain applications.

As used throughout this document, the term “couple” and or “coupled” refers to any direct or indirect communication between two or more elements, whether or not those elements are physically connected to one another. In this disclosure, the term “damage” refers to affecting a tissue or sample so as to render the tissue or sample inoperable. For instance, if a particular tissue normally emits certain signaling chemicals, then by “damaging” the tissue is meant that the tissue reduces or no longer emits that certain signaling chemical. The term “damage” and or “damaged” may include ablation, melting, charring, killing, or simply incapacitating the chemical emissions from the particular tissue or sample. In one embodiment, histology or histochemical analysis may be used to determine whether a tissue or sample has been damaged.

As used throughout this disclosure, the term “spectroscopy” means that a tissue or sample is inspected by comparing different features, such as wavelength (or frequency), spatial location, transmission, absorption, reflectivity, scattering, refractive index, or opacity. In one embodiment, “spectroscopy” may mean that the wavelength of the light source is varied, and the transmission, absorption or reflectivity of the tissue or sample is measured as a function of wavelength. In another embodiment, “spectroscopy” may mean that the wavelength dependence of the transmission, absorption or reflectivity is compared between different spatial locations on a tissue or sample. As an illustration, the “spectroscopy” may be performed by varying the wavelength of the light source, or by using a broadband light source and analyzing the signal using a spectrometer, wavemeter, or optical spectrum analyzer.

As used throughout this document, the term “fiber laser” refers to a laser or oscillator that has as an output light or an optical beam, wherein at least a part of the laser comprises an optical fiber. For instance, the fiber in the “fiber laser” may comprise one of or a combination of a single mode fiber, a multi-mode fiber, a mid-infrared fiber, a photonic crystal fiber, a doped fiber, a gain fiber, or, more generally, an approximately cylindrically shaped waveguide or light-pipe. In one embodiment, the gain fiber may be doped with rare earth material, such as ytterbium, erbium, and/or thulium. In another embodiment, the infrared fiber may comprise one or a combination of fluoride fiber, ZBLAN fiber, chalcogenide fiber, tellurite fiber, or germanium doped fiber. In yet another embodiment, the single mode fiber may include standard single-mode fiber, dispersion shifted fiber, non-zero dispersion shifted fiber, high-nonlinearity fiber, and small core size fibers.

As used throughout this disclosure, the term “pump laser” refers to a laser or oscillator that has as an output light or an optical beam, wherein the output light or optical beam may be coupled to a gain medium to excite the gain medium, which in turn may amplify another input optical signal or beam. In one particular example, the gain medium may be a doped fiber, such as a fiber doped with ytterbium, erbium, and/or thulium. In another embodiment, the gain medium may be a fused silica fiber or a fiber with a Raman effect from the glass. In one embodiment, the “pump laser” may be a fiber laser, a solid state laser, a laser involving a nonlinear crystal, an optical parametric oscillator, a semiconductor laser, or a plurality of semiconductor lasers that may be multiplexed together. In another embodiment, the “pump laser” may be coupled to the gain medium by using a fiber coupler, a dichroic mirror, a multiplexer, a wavelength division multiplexer, a grating, or a fused fiber coupler.

As used throughout this document, the term “super-continuum” and/or “supercontinuum” and/or “SC” refers to a broadband light beam or output that comprises a plurality of wavelengths. In a particular example, the plurality of wavelengths may be adjacent to one-another, so that the spectrum of the light beam or output appears as a continuous band when measured with a spectrometer. In one embodiment, the broadband light beam may have a bandwidth of at least 10 nm. In another embodiment, the “super-continuum” may be generated through nonlinear optical interactions in a medium, such as an optical fiber or nonlinear crystal. For example, the “super-continuum” may be generated through one or a combination of nonlinear activities such as four-wave mixing, the Raman effect, modulational instability, and self-phase modulation.

As used throughout this disclosure, the terms “optical light” and/or “optical beam” and or “light beam” refer to photons or light transmitted to a particular location in space. The “optical light” and or “optical beam” and/or “light beam” may be modulated or unmodulated, which also means that they may or may not contain information. In one embodiment, the “optical light” and/or “optical beam” and/or “light beam” may originate from a fiber, a fiber laser, a laser, a light emitting diode, a lamp, a pump laser, or a light source.

As used throughout this document, the terms “near” or “about” or the symbol “˜” refer to one or more wavelengths of light with wavelengths around the stated wavelength to accomplish the function described. For example, “near 1720 nm” may include wavelengths of between about 1680 nm and 1760 nm. In one embodiment, the term “near 1720 nm” refers to one or more wavelengths of light with a wavelength value anywhere between approximately 1700 nm and 1740 nm. Similarly, as used throughout this document, the term “near 1210 nm” refers to one or wavelengths of light with a wavelength value anywhere between approximately 1170 nm and 1250 nm. In one embodiment, the term “near 1210 nm” refers to one or more wavelengths of light with a wavelength value anywhere between approximately 1190 nm and 1230 nm.

Different light sources may be selected for the infrared based on the needs of the application. Some of the features for selecting a particular light source include power or intensity, wavelength range or bandwidth, spatial or temporal coherence, spatial beam quality for focusing or transmission over long distance, and pulse width or pulse repetition rate. Depending on the application, lamps, light emitting diodes (LEDs), laser diodes (LD's), tunable LD's, super-luminescent laser diodes (SLDs), fiber lasers or super-continuum (SC) sources may be advantageously used. Also, different fibers may be used for transporting the light, such as fused silica fibers, plastic fibers, mid-infrared fibers (e.g., tellurite, chalcogenides, fluorides, ZBLAN, etc.), photonic crystal fibers, or a hybrid of these fibers.

In one embodiment, LED's can be used that have a higher power level in the infrared wavelength range. LED's produce an incoherent beam, but the power level can be higher than a lamp and with higher energy efficiency. Also, the LED output may more easily be modulated, and the LED provides the option of continuous wave or pulsed mode of operation. LED's are solid state components that emit a wavelength band that is of moderate width, typically between about 20 nm to 40 nm. There are also so-called super-luminescent LEDs that may even emit over a much wider wavelength range. In another embodiment, a wide band light source may be constructed by combining different LEDs that emit in different wavelength bands, some of which could preferably overlap in spectrum. One advantage of LEDs as well as other solid state components is the compact size that they may be packaged into.

In yet another embodiment, various types of laser diodes may be used in the infrared wavelength range. Just as LEDs may be higher in power but narrower in wavelength emission than lamps and thermal sources, the LDs may be yet higher in power but yet narrower in wavelength emission than LEDs. Different kinds of LDs may be used, including Fabry-Perot LDs, distributed feedback (DFB) LDs, distributed Bragg reflector (DBR) LDs. A plurality of LDs may be spatially multiplexed, polarization multiplexed, wavelength multiplexed, or a combination of these multiplexing methods. Also, the LDs may be fiber pig-tailed or have one or more lenses on the output to collimate or focus the light. Another advantage of LDs is that they may be packaged compactly and may have a spatially coherent beam output. Moreover, tunable LDs that can tune over a range of wavelengths are also available. The tuning may be done by varying the temperature, or electrical current may be used in particular structures such as distributed Bragg reflector (DBR) LDs. In another embodiment, external cavity LDs may be used that have a tuning element, such as a fiber grating or a bulk grating, in the external cavity.

In another embodiment, super-luminescent laser diodes may provide higher power as well as broad bandwidth. An SLD is typically an edge emitting semiconductor light source based on super-luminescence (e.g., this could be amplified spontaneous emission). SLDs combine the higher power and brightness of LDs with the low coherence of conventional LEDs, and the emission band for SLD's may be 5 nm to 100 nm wide, preferably in the 60 nm to 100 nm range for some applications. Although currently SLDs are commercially available in the wavelength range of approximately 400 nm to 1700 nm, SLDs could and may in the future be made the cover a broader region of the infrared.

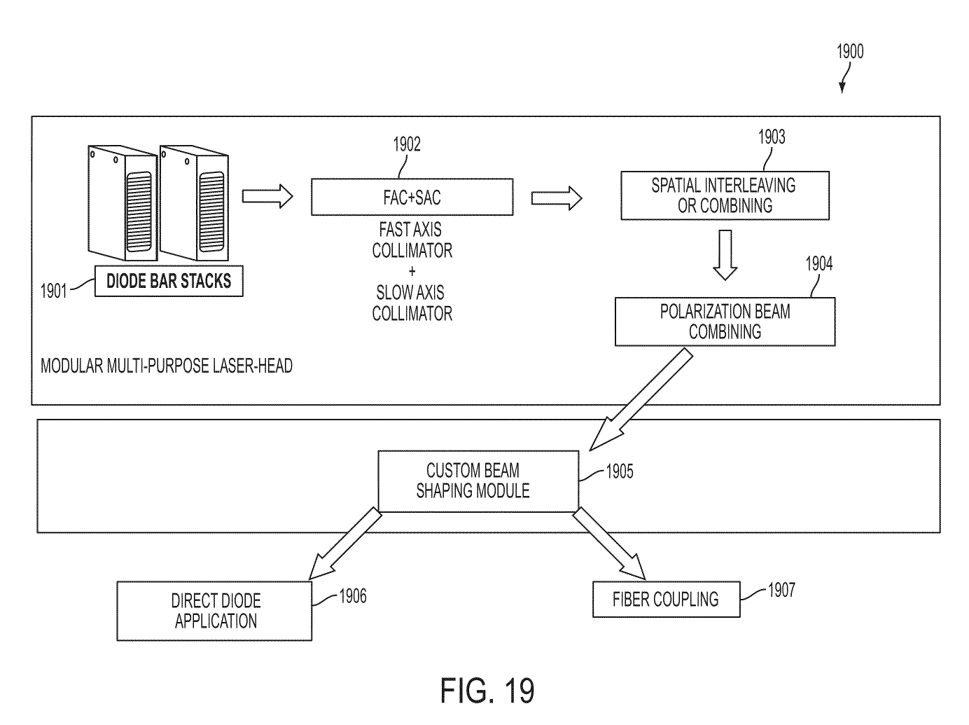

In yet another embodiment, high power LDs for either direct excitation or to pump fiber lasers and SC light sources may be constructed using one or more laser diode bar stacks. As an example, FIG. 19 shows an example of the block diagram 1900 or building blocks for constructing the high power LDs. In this embodiment, one or more diode bar stacks 1901 may be used, where the diode bar stack may be an array of several single emitter LDs. Since the fast axis (e.g., vertical direction) may be nearly diffraction limited while the slow-axis (e.g., horizontal axis) may be far from diffraction limited, different collimators 1902 may be used for the two axes.

Then, the brightness may be increased by spatially combining the beams from multiple stacks 1903. The combiner may include spatial interleaving, it may include wavelength multiplexing, or it may involve a combination of the two. Different spatial interleaving schemes may be used, such as using an array of prisms or mirrors with spacers to bend one array of beams into the beam path of the other. In another embodiment, segmented mirrors with alternate high-reflection and anti-reflection coatings may be used. Moreover, the brightness may be increased by polarization beam combining 1904 the two orthogonal polarizations, such as by using a polarization beam splitter. In a particular embodiment, the output may then be focused or coupled into a large diameter core fiber. As an example, typical dimensions for the large diameter core fiber range from diameters of approximately 100 microns to 400 microns or more. Alternatively or in addition, a custom beam shaping module 1905 may be used, depending on the particular application. For example, the output of the high power LD may be used directly 1906, or it may be fiber coupled 1907 to combine, integrate, or transport the high power LD energy. These high power LDs may grow in importance because the LD powers can rapidly scale up. For example, instead of the power being limited by the power available from a single emitter, the power may increase in multiples depending on the number of diodes multiplexed and the size of the large diameter fiber. Although FIG. 19 is shown as one embodiment, some or all of the elements may be used in a high power LD, or additional elements may also be used.

### Infrared Super-Continuum Lasers

Each of the light sources described above have particular strengths, but they also may have limitations. For example, there is typically a trade-off between wavelength range and power output. Also, sources such as lamps, thermal sources, and LEDs produce incoherent beams that may be difficult to focus to a small area and may have difficulty propagating for long distances. An alternative source that may overcome some of these limitations is an SC light source. Some of the advantages of the SC source may include high power and intensity, wide bandwidth, spatially coherent beam that can propagate nearly transform limited over long distances, and easy compatibility with fiber delivery.

Supercontinuum lasers may combine the broadband attributes of lamps with the spatial coherence and high brightness of lasers. By exploiting a modulational instability initiated supercontinuum (SC) mechanism, an all-fiber-integrated SC laser with no moving parts may be built using commercial-off-the-shelf (COTS) components. Moreover, the fiber laser architecture may be a platform where SC in the visible, near-infrared/SWIR, or mid-IR can be generated by appropriate selection of the amplifier technology and the SC generation fiber. But until now, SC lasers were used primarily in laboratory settings since typically large, table-top, mode-locked lasers were used to pump nonlinear media such as optical fibers to generate SC light. However, those large pump lasers may now be replaced with diode lasers and fiber amplifiers that gained maturity in the telecommunications industry.

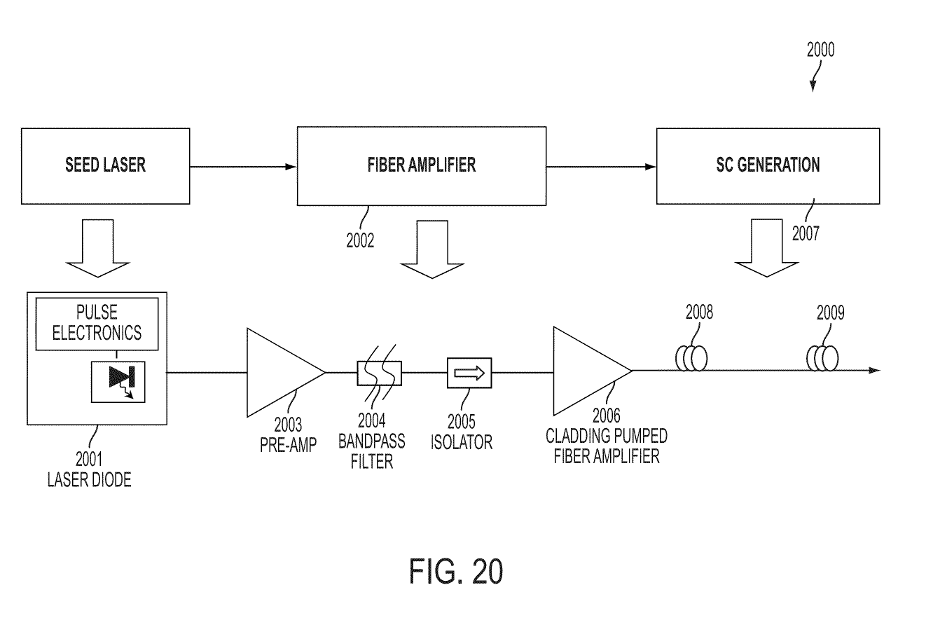

In one embodiment, an all-fiber-integrated, high-powered SC light source 2000 may be elegant for its simplicity (FIG. 20). The light may be first generated from a seed laser diode 2001. For example, the seed LD 2001 may be a distributed feedback (DFB) laser diode with a wavelength near 1542 nm or 1550 nm, with approximately 0.5-2.0 ns pulsed output, and with a pulse repetition rate between one kilohertz and about 100 MHz or more. The output from the seed laser diode may then be amplified in a multiple-stage fiber amplifier 2002 comprising one or more gain fiber segments. In a particular embodiment, the first stage pre-amplifier 2003 may be designed for optimal noise performance. For example, the pre-amplifier 2003 may be a standard erbium-doped fiber amplifier or an erbium/ytterbium doped cladding pumped fiber amplifier. Between amplifier stages 2003 and 2006, it may be advantageous to use band-pass filters 2004 to block amplified spontaneous emission and isolators 2005 to prevent spurious reflections. Then, the power amplifier stage 2006 may use a cladding-pumped fiber amplifier that may be optimized to minimize nonlinear distortion. The power amplifier fiber 2006 may also be an erbium-doped fiber amplifier, if only low or moderate power levels are to be generated.

The SC generation 2007 may occur in the relatively short lengths of fiber that follow the pump laser. Exemplary SC fiber lengths may range from a few millimeters to 100 m or more. In one embodiment, the SC generation may occur in a first fiber 2008 where the modulational-instability initiated pulse break-up occurs primarily, followed by a second fiber 2009 where the SC generation and spectral broadening occurs primarily.

In one embodiment, one or two meters of standard single-mode fiber (SMF) after the power amplifier stage may be followed by several meters of SC generation fiber. For this example, in the SMF the peak power may be several kilowatts and the pump light may fall in the anomalous group-velocity dispersion regime—often called the soliton regime. For high peak powers in the dispersion regime, the nanosecond pulses may be unstable due to a phenomenon known as modulational instability, which is basically parametric amplification in which the fiber nonlinearity helps to phase match the pulses. As a consequence, the nanosecond pump pulses may be broken into many shorter pulses as the modulational instability tries to form soliton pulses from the quasi-continuous-wave background. Although the laser diode and amplification process starts with approximately nanosecond-long pulses, modulational instability in the short length of SMF fiber may form approximately 0.5 ps to several-picosecond-long pulses with high intensity. Thus, the few meters of SMF fiber may result in an output similar to that produced by mode-locked lasers, except in a much simpler and cost-effective manner.

The short pulses created through modulational instability may then be coupled into a nonlinear fiber for SC generation. The nonlinear mechanisms leading to broadband SC may include four-wave mixing or self-phase modulation along with the optical Raman effect. Since the Raman effect is self-phase-matched and shifts light to longer wavelengths by emission of optical photons, the SC may spread to longer wavelengths very efficiently. The short-wavelength edge may arise from four-wave mixing, and often times the short wavelength edge may be limited by increasing group-velocity dispersion in the fiber. In many instances, if the particular fiber used has sufficient peak power and SC fiber length, the SC generation process may fill the long-wavelength edge up to the transmission window.

Mature fiber amplifiers for the power amplifier stage 2006 include ytterbium-doped fibers (near 1060 nm), erbium-doped fibers (near 1550 nm), erbium/ytterbium-doped fibers (near 1550 nm), or thulium-doped fibers (near 2000 nm). In various embodiments, candidates for SC fiber 2009 include fused silica fibers (for generating SC between 0.8-2.7 μm), mid-IR fibers such as fluorides, chalcogenides, or tellurites (for generating SC out to 4.5 μm or longer), photonic crystal fibers (for generating SC between 0.4 and 1.7 μm), or combinations of these fibers. Therefore, by selecting the appropriate fiber-amplifier doping for 2006 and nonlinear fiber 2009, SC may be generated in the visible, near-IR/SWIR, or mid-IR wavelength region.

The configuration 2000 of FIG. 20 is just one particular example, and other configurations can be used and are intended to be covered by this disclosure. For example, further gain stages may be used, and different types of lossy elements or fiber taps may be used between the amplifier stages. In another embodiment, the SC generation may occur partially in the amplifier fiber and in the pig-tails from the pump combiner or other elements. In yet another embodiment, polarization maintaining fibers may be used, and a polarizer may also be used to enhance the polarization contrast between amplifier stages. Also, not discussed in detail are many accessories that may accompany this set-up, such as driver electronics, pump laser diodes, safety shut-offs, and thermal management and packaging.

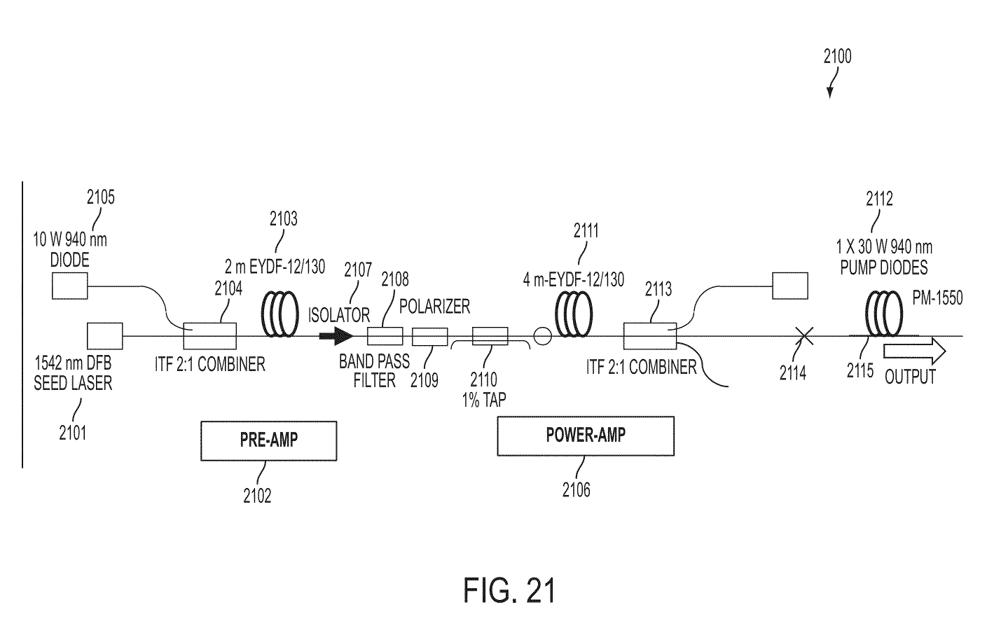

In one embodiment, one example of the SC laser that operates in the short wave infrared (SWIR) is illustrated in FIG. 21. This SWIR SC source 2100 produces an output of up to approximately 5 W over a spectral range of about 1.5 microns to 2.4 microns, and this particular laser is made out of polarization maintaining components. The seed laser 2101 is a distributed feedback (DFB) laser operating near 1542 nm producing approximately 0.5 nsec pulses at an about 8 MHz repetition rate. The pre-amplifier 2102 is forward pumped and uses about 2 m length of erbium/ytterbium cladding pumped fiber 2103 (often also called dual-core fiber) with an inner core diameter of 12 microns and outer core diameter of 130 microns. The pre-amplifier gain fiber 2103 is pumped using a 10 W 940 nm laser diode 2105 that is coupled in using a fiber combiner 2104.

In this particular 5 W unit, the mid-stage between amplifier stages 2102 and 2106 comprises an isolator 2107, a band-pass filter 2108, a polarizer 2109 and a fiber tap 2110. The power amplifier 2106 uses a 4 m length of the 12/130 micron erbium/ytterbium doped fiber 2111 that is counter-propagating pumped using one or more 30 W 940 nm laser diodes 2112 coupled in through a combiner 2113. An approximately 1-2 meter length of the combiner pig-tail helps to initiate the SC process, and then a length of PM-1550 fiber 2115 (polarization maintaining, single-mode, fused silica fiber optimized for 1550 nm) is spliced 2114 to the combiner output.

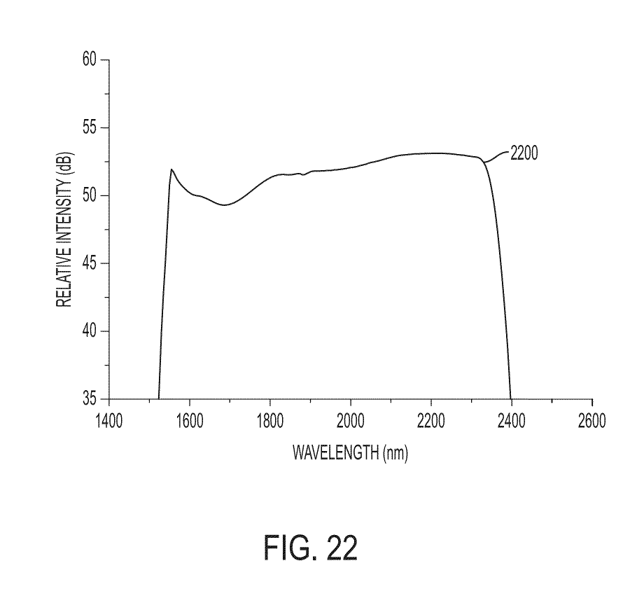

If an approximately 10 m length of output fiber is used, then the resulting output spectrum 2200 is shown in FIG. 22. The details of the output spectrum 2200 depend on the peak power into the fiber, the fiber length, and properties of the fiber such as length and core size, as well as the zero dispersion wavelength and the dispersion properties. For example, if a shorter length of fiber is used, then the spectrum actually reaches to longer wavelengths (e.g., a 2 m length of SC fiber broadens the spectrum to about 2500 nm). Also, if extra-dry fibers are used with less O-H content, then the wavelength edge may also reach to a longer wavelength. To generate more spectrum toward the shorter wavelengths, the pump wavelength (in this case ˜1542 nm) should be close to the zero dispersion wavelength in the fiber. For example, by using a dispersion shifted fiber or so-called non-zero dispersion shifted fiber, the short wavelength edge may shift to shorter wavelengths.

Although one particular example of a 5 W SWIR-SC implementation has been described, different components, different fibers, and different configurations may also be used consistent with this disclosure. For instance, another embodiment of the similar configuration 2100 in FIG. 21 may be used to generate high powered SC between approximately 1060 nm and 1800 nm. For this embodiment, the seed laser 2101 may be a 1064 nm distributed feedback (DFB) laser diode, the pre-amplifier gain fiber 2103 may be a ytterbium-doped fiber amplifier with 10/125 microns dimensions, and the pump laser 2105 may be a 10 W 915 nm laser diode. A mode field adapter may be included in the mid-stage, in addition to the isolator 2107, band pass filter 2108, polarizer 2109 and tap 2110. The gain fiber 2111 in the power amplifier may be a 20 m length of ytterbium-doped fiber with 25/400 microns dimension. The pump 2112 for the power amplifier may be up to six pump diodes providing 30 W each near 915 nm. For this much pump power, the output power in the SC may be as high as 50 W or more.

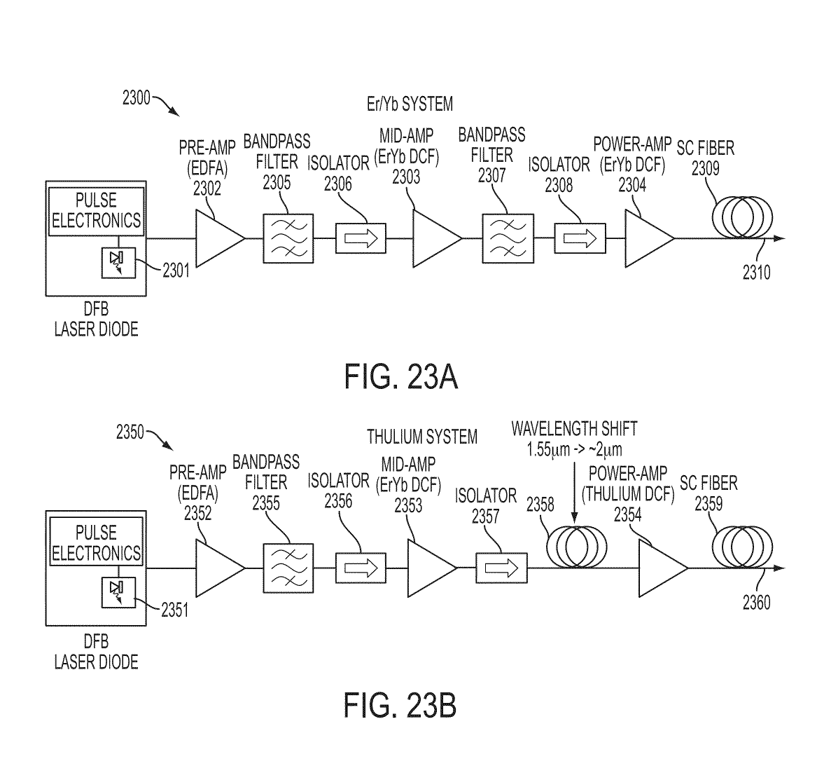

In an alternate embodiment, it may be desirous to generate high power SWIR SC over 1.4-1.8 microns and separately 2-2.5 microns (the window between 1.8 and 2 microns may be less important due to the strong water and atmospheric absorption). For example, the top SC source of FIG. 23 can lead to bandwidths ranging from about 1400 nm to 1800 nm or broader, while the lower SC source of FIG. 23 can lead to bandwidths ranging from about 1900 nm to 2500 nm or broader. Since these wavelength ranges are shorter than about 2500 nm, the SC fiber can be based on fused silica fiber. Exemplary SC fibers include standard single-mode fiber (SMF), high-nonlinearity fiber, high-NA fiber, dispersion shifted fiber, dispersion compensating fiber, and photonic crystal fibers. Non-fused-silica fibers can also be used for SC generation, including chalcogenides, fluorides, ZBLAN, tellurites, and germanium oxide fibers.

In one embodiment, the top of FIG. 23 illustrates a block diagram for an SC source 2300 capable of generating light between approximately 1400 nm and 1800 nm or broader. As an example, a pump fiber laser similar to FIG. 21 can be used as the input to a SC fiber 2309. The seed laser diode 2301 can comprise a DFB laser that generates, for example, several milliwatts of power around 1542 nm or 1553 nm. The fiber pre-amplifier 2302 can comprise an erbium-doped fiber amplifier or an erbium/ytterbium doped double clad fiber. In this example a mid-stage amplifier 2303 can be used, which can comprise an erbium/ytterbium doped double-clad fiber. A bandpass filter 2305 and isolator 2306 may be used between the pre-amplifier 2302 and mid-stage amplifier 2303. The power amplifier stage 2304 can comprise a larger core size erbium/ytterbium doped double-clad fiber, and another bandpass filter 2307 and isolator 2308 can be used before the power amplifier 2304. The output of the power amplifier can be coupled to the SC fiber 2309 to generate the SC output 2310. This is just one exemplary configuration for an SC source, and other configurations or elements may be used consistent with this disclosure.

In yet another embodiment, the bottom of FIG. 23 illustrates a block diagram for an SC source 2350 capable of generating light exemplary between approximately 1900 nm and 2500 nm or broader. As an example, the seed laser diode 2351 can comprise a DFB or DBR laser that generates, for example, several milliwatts of power around 1542 nm or 1553 nm. The fiber pre-amplifier 2352 can comprise an erbium-doped fiber amplifier or an erbium/ytterbium doped double-clad fiber. In this example a mid-stage amplifier 2353 can be used, which can comprise an erbium/ytterbium doped double-clad fiber. A bandpass filter 2355 and isolator 2356 may be used between the pre-amplifier 2352 and mid-stage amplifier 2353. The power amplifier stage 2354 can comprise a thulium doped double-clad fiber, and another isolator 2357 can be used before the power amplifier 2354. Note that the output of the mid-stage amplifier 2353 can be approximately near 1550 nm, while the thulium-doped fiber amplifier 2354 can amplify wavelengths longer than approximately 1900 nm and out to about 2100 nm. Therefore, for this configuration wavelength shifting may be required between 2353 and 2354. In one embodiment, the wavelength shifting can be accomplished using a length of standard single-mode fiber 2358, which can have exemplary lengths between approximately 5 meters and 50 meters. The output of the power amplifier 2354 can be coupled to the SC fiber 2359 to generate the SC output 2360. This is just one exemplary configuration for an SC source, and other configurations or elements can be used consistent with this disclosure. For example, the various amplifier stages can comprise different amplifier types, such as erbium doped fibers, ytterbium doped fibers, erbium/ytterbium co-doped fibers and thulium doped fibers. One advantage of the SC lasers illustrated in FIGS. 20-23 are that they may use all-fiber components, so that the SC laser can be all-fiber, monolithically integrated with no moving parts. The all-integrated configuration can consequently be robust and reliable.

FIGS. 20-23 are examples of SC light sources that may advantageously be used for SWIR light generation in various medical diagnostic and therapeutic applications. However, many other versions of the SC light sources may also be made that are intended to also be covered by this disclosure. For example, the SC generation fiber could be pumped by a mode-locked laser, a gain-switched semiconductor laser, an optically pumped semiconductor laser, a solid state laser, other fiber lasers, or a combination of these types of lasers. Also, rather than using a fiber for SC generation, either a liquid or a gas cell might be used as the nonlinear medium in which the spectrum is to be broadened.

Even within the all-fiber versions illustrated such as in FIG. 21, different configurations could be used consistent with the disclosure. In an alternate embodiment, it may be desirous to have a lower cost version of the SWIR SC laser of FIG. 21. One way to lower the cost could be to use a single stage of optical amplification, rather than two stages, which may be feasible if lower output power is required or the gain fiber is optimized. For example, the pre-amplifier stage 2102 might be removed, along with at least some of the mid-stage elements. In yet another embodiment, the gain fiber could be double passed to emulate a two stage amplifier. In this example, the pre-amplifier stage 2102 might be removed, and perhaps also some of the mid-stage elements. A mirror or fiber grating reflector could be placed after the power amplifier stage 2106 that may preferentially reflect light near the wavelength of the seed laser 2101. If the mirror or fiber grating reflector can transmit the pump light near 940 nm, then this could also be used instead of the pump combiner 2113 to bring in the pump light 2112. The SC fiber 2115 could be placed between the seed laser 2101 and the power amplifier stage 2106 (SC is only generated after the second pass through the amplifier, since the power level may be sufficiently high at that time). In addition, an output coupler may be placed between the seed laser diode 2101 and the SC fiber, which now may be in front of the power amplifier 2106. In a particular embodiment, the output coupler could be a power coupler or divider, a dichroic coupler (e.g., passing seed laser wavelength but outputting the SC wavelengths), or a wavelength division multiplexer coupler. This is just one further example, but a myriad of other combinations of components and architectures could also be used for SC light sources to generate SWIR light that are intended to be covered by this disclosure.

### Fiber Lasers Based on Cascaded Raman Shifting

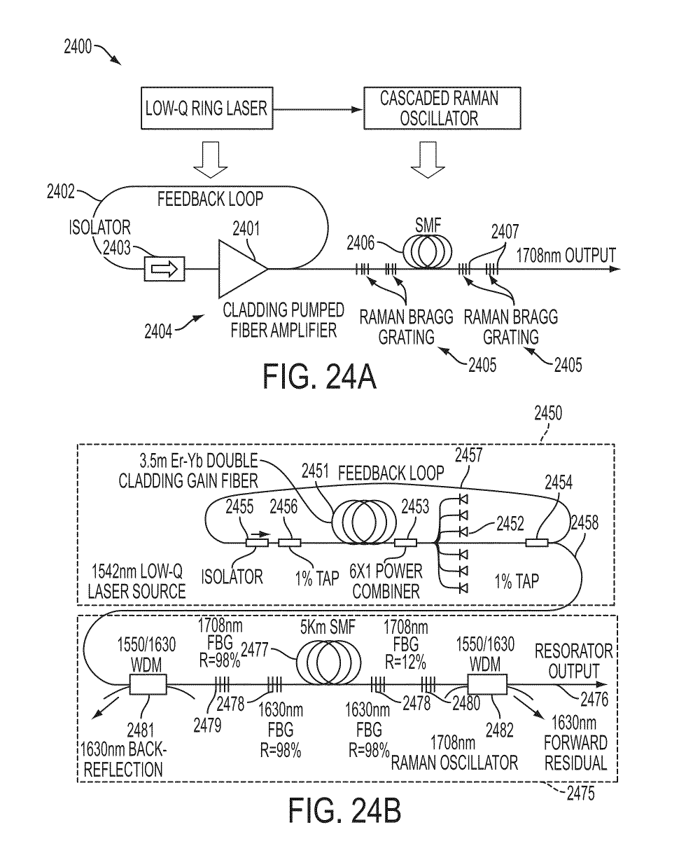

For therapeutic applications, it may be desirable to generate laser power with high spectral density in a narrower wavelength range. As an alternative to multiplexed laser diodes such as in FIG. 19, one option may be to use fiber lasers based on the cascaded Raman wavelength shifting. FIG. 24A illustrates a block diagram of one embodiment of an infrared fiber laser 2400 operating near 1720 nm. One advantage of such a configuration can be that all of the fiber parts can be spliced together to result in an all-fiber, monolithically integrated, no moving parts light source. In this particular example, the pump fiber laser 2404 can be a cladding pumped fiber amplifier 2401 with a feedback loop 2402 around the amplifier to cause lasing. In one non-limiting example, an isolator 2403 can be placed in the ring cavity of the pump laser to cause the lasing to be unidirectional. In this case, the cladding pumped fiber amplifier 2401 can be an erbium/ytterbium doped amplifier operating near 1550 nm. The pump laser light can then be coupled to a cascaded Raman oscillator 2405, where the fiber 2406 can be a single-mode fiber and two sets of Bragg gratings 2407 can be used to wavelength shift out to near 1720 nm.

In one embodiment, a specific example of the infrared fiber laser operating at approximately 1708 nm is shown in detail in FIG. 24B. The top part of the figure illustrates one embodiment of the pump fiber laser 2450 details, while the bottom part of the figure illustrates one embodiment of the cascaded Raman oscillator 2475 details. In the pump fiber laser, the gain fiber 2451 can be an erbium-ytterbium doped, double clad fiber, for example. In one embodiment, the length of the gain fiber can be between about 3 meters and 6 meters. One or more pump laser diodes 2452 can be used to excite the gain fiber 2451. In one embodiment, the pump lasers 2452 can operate at wavelengths between approximately 935 nm and 980 nm, and between 4 and 18 pump laser diodes may be used. The one or more pump laser diodes 2452 can be combined using a power combiner 2453, and then the combined pump laser diode power can be coupled to the gain fiber 2451. In this particular example, the pump laser diodes 2452 can be coupled into the gain fiber 2451 in a counter-propagating direction to the signal in the oscillator. However, the pump laser diodes could also co-propagate with the direction of the signal in the oscillator. After the pump combiner 2453, a part of the output of the gain fiber can be separated at a power tap 2454 and then fed back to the input using a feedback loop fiber 2457. In the loop, an isolator 2455 can also be inserted to permit unidirectional operation and lasing (in this particular example, the pump fiber laser 2450 resonates in a counter-clockwise direction). Other elements may also be inserted into the ring cavity, such as additional taps 2456. Although one particular example of a pump fiber laser 2450 is described, any number of changes in elements or their positions can be made consistent with this disclosure.

The bottom of FIG. 24B illustrates one embodiment of a cascaded Raman oscillator 2475 for shifting the pump fiber laser output wavelength to a longer signal wavelength 2476. The center of the oscillator is a Raman gain fiber 2477, which in this particular embodiment can be a standard single mode fiber (SMF). The length of the SMF can be in the range of about 300 m to 10 km, and as an example in this embodiment may be closer to approximately 5 km. Any number of fiber types, including high nonlinearity fibers, mid-infrared fibers, high numerical aperture fibers, or photonic crystal fibers, can be used consistent with this disclosure. The Raman gain fiber 2477 can be surrounded by a plurality of fiber Bragg gratings (FBG), 2478, 2479 and 2480. In this particular embodiment, two cascaded Raman orders are used to transfer the pump output wavelength 2458 near 1550 nm to the longer signal wavelength near 1708 nm. Hence, in FIG. 24B there can be two sets of fiber Bragg gratings (FBR).

As an example, the inner grating set 2478 can be designed to provide high reflectivity near 1630 nm. The reflectivity can be in the range of about 70% t to 90%, but in this particular embodiment can be closer to 98%. The outer grating set 2479 and 2480 can be designed to reflect light near 1708 nm (i.e., the desired longer signal wavelength). The first fiber Bragg grating 2479 can have high reflectivity, for example in the range of 70 to 90 percent, but more preferably is closer to 98%. The second fiber Bragg grating 2480 also serves as the output coupler, and hence should have a lower reflectivity value. As an example, the reflectivity of grating 2480 can be in the range of 8% to 50%, and is preferably closer to 12%.

Moreover, to remove the residual shifted pump light from the first or intermediate orders of Raman shifting, WDM couplers can be used surrounding the oscillator, such as 2481 and 2482. In this particular embodiment, the WDM couplers 2481 and 2482 are 1550/1630 couplers (i.e., couplers that pass light near 1550 nm but that couple across or out wavelengths near 1630 nm). Such couplers can help to avoid feedback into the pump fiber laser 2450 as well as minimize the residual intermediate orders in the longer signal wavelength 2476. It may also be beneficial to add an isolator between the pump fiber laser 2450 and the cascaded Raman oscillator 2475 to minimize the effects of feedback. Although one specific example is provided for the cascaded Raman oscillator 2475, any number of changes in the components or values or additional components can be made and are intended to be covered in this disclosure.

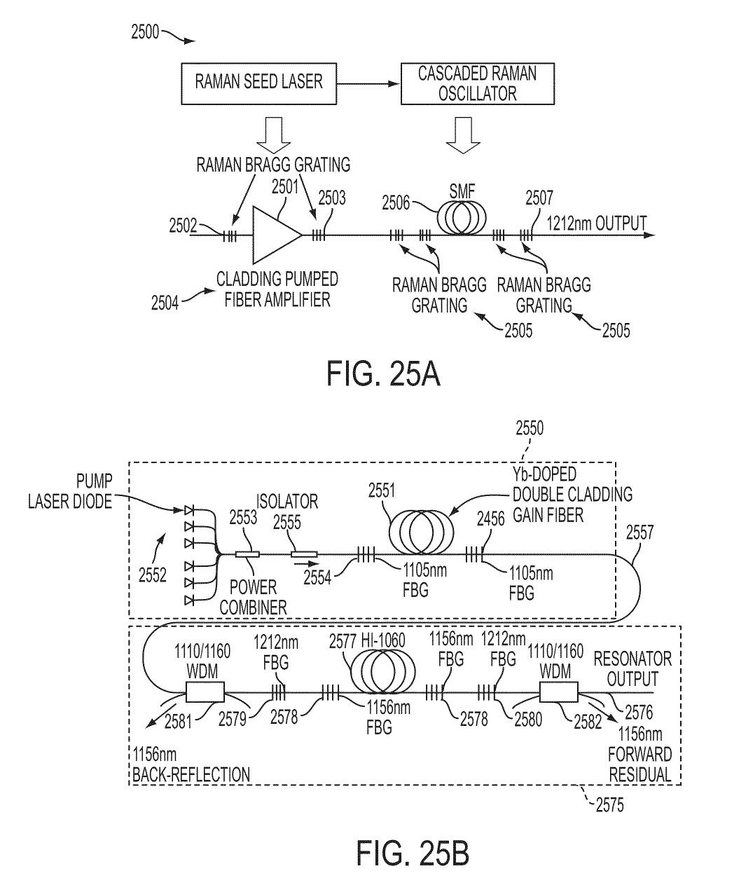

FIG. 25A illustrates a block diagram of yet another embodiment of an infrared fiber laser 2500 that operates near 1212 nm. Whereas FIG. 24 uses a ring cavity pump fiber laser, FIG. 25 uses a linear cavity pump fiber laser. Either of these configurations or other versions of the pump fiber laser can be used consistent with this disclosure. In this particular example, the pump fiber laser 2504 can be a cladding pumped fiber amplifier 2501 surrounded by fiber Bragg gratings 2502 and 2503 around the amplifier to cause lasing. In this case, the cladding pumped fiber amplifier 2501 can be a ytterbium doped amplifier operating approximately in the wavelength range between 1050 nm and 1120 nm. The pump laser light can then be coupled to a cascaded Raman oscillator 2505, where the fiber 2506 can be a single-mode fiber and two sets of Bragg gratings 2507 are used to wavelength shift out to near 1212 nm.

In yet another embodiment, a specific example of the infrared fiber laser operating at approximately 1212 nm is shown in detail in FIG. 25B. The top part of the figure illustrates one embodiment of the pump fiber laser 2550 details, while the bottom part of the figure illustrates one embodiment of the cascaded Raman oscillator 2575 details. In the pump fiber laser, the gain fiber 2551 can be a ytterbium doped, double clad fiber, for example. In one embodiment, the length of the gain fiber can be between about 3 meters and 10 meters. One or more pump laser diodes 2552 can be used to excite the gain fiber 2551. In one embodiment, the pump lasers 2552 can operate at wavelengths between approximately 850 nm and 980 nm, and between 2 and 18 pump laser diodes may be used. The one or more pump laser diodes 2552 can be combined using a power combiner 2553, and then the combined pump laser diode power can be coupled to the gain fiber 2551. After the pump combiner 2553, it may be beneficial to use one or more isolators 2555 to avoid feedback into the pump laser diodes 2552.

The pump fiber laser can be formed by using a set of gratings 2554 and 2556 around the gain fiber 2551. In one embodiment, the fiber Bragg gratings 2554 and 2556 can have reflecting at a wavelength near 1105 nm. The reflectivity of 2554 can be in the range of 70% to 90%, and in this particular embodiment can be closer to 98%. The second fiber Bragg grating 2556 can also serve as the output coupler, and hence may have a lower reflectivity value. As an example, the reflectivity of grating 2556 can be in the range of 5% to 50%, but is preferably closer to 10% in this embodiment. Other elements may also be inserted into the linear resonator cavity, such as additional taps. Although one particular example of a pump fiber laser 2550 is described, any number of changes in elements or their positions can be made consistent with this disclosure.

The bottom of FIG. 25B illustrates one embodiment of a cascaded Raman oscillator 2575 for shifting the pump fiber laser output wavelength to a longer signal wavelength 2576. The center of the oscillator is a Raman gain fiber 2577, which in this particular embodiment can be a HI-1060 fiber, which operates at a single spatial mode at the wavelengths of the ytterbium amplifier. The length of the Raman gain fiber 2577 can be in the range of 300 m to 10 km, and as an example in this embodiment may be closer to approximately 1 km. Any number of fiber types, including high nonlinearity fibers, mid-infrared fibers, high numerical aperture fibers, or photonic crystal fibers, can be used consistent with this disclosure. The Raman gain fiber 2577 can be surrounded by a plurality of fiber Bragg gratings FBG, 2578, 2579 and 2580. In this particular embodiment, two cascaded Raman orders are used to transfer the pump output wavelength 2557 near 1105 nm to the longer signal wavelength near 1212 nm. Hence, in FIG. 25B there can be two sets of fiber Bragg gratings.

As an example, the inner grating set 2578 can be designed to provide high reflectivity near 1156 nm. The reflectivity can be in the range of 70% to 90%, and in this particular embodiment can be closer to 99%. The outer grating set 2579 and 2580 can be designed to reflect light near 1212 nm (i.e., the desired longer signal wavelength). The first fiber Bragg grating 2579 can have high reflectivity, for example in the range of 70% to 90%, but in this embodiment is closer to 99%. The second fiber Bragg grating 2580 can also serve as the output coupler, and hence may have a lower reflectivity value. As an example, the reflectivity of grating 2580 can be in the range of 8% to 50%, but is closer to 25% in this embodiment.

Moreover, to remove the residual shifted pump light from the first or intermediate orders of Raman shifting, WDM couplers can be used surrounding the oscillator, such as 2581 and 2582. In this particular embodiment, the WDM couplers 2581 and 2582 are 1100/1160 couplers (i.e., couplers that pass light near 1100 nm but that couple across or out wavelengths near 1160 nm). Such couplers can help to avoid feedback into the pump fiber laser 2550 as well as minimize the residual intermediate orders in the longer signal wavelength 2576. It may also be beneficial to add an isolator between the pump fiber laser 2550 and the cascaded Raman oscillator 2575 to minimize the effects of feedback. Although one specific example is provided for the cascaded Raman oscillator 2575, any number of changes in the components or values or additional components can be made and are intended to be covered in this disclosure.

### Laser Beam Output Parameters

The laser beam output that may be used in the healthcare, medical or bio-technology applications can have a number of parameters, including wavelength, power, energy or fluence, spatial spot size, and pulse temporal shape and repetition rate. Some exemplary ranges for these parameters and some of the criteria for selecting the ranges are discussed herein. These are only meant to be exemplary ranges and considerations, and the particular combination used may depend on the details and goals of the desired procedure.

Whereas it may be advantageous in a diagnostic procedure to use a broadband laser such as a super-continuum source, for various therapeutic procedures the wavelength for the laser may be selected on the basis of a number of considerations, such as penetration depth or absorption in a particular type of tissue or water. In yet another embodiment, it may be advantageous to have the laser wavelength fall in the so-called eye-safe wavelength range. For instance, wavelengths longer than approximately 1400 nm can fall within the eye safe window. So, from an eye safety consideration there may be an advantage of using the wavelength window near 1720 nm rather than the window near 1210 nm. Thus, some of the considerations in selecting the laser wavelength range from selective tissue absorption, water absorption and scattering loss, penetration depth into tissue and eye safe operation.

Another parameter for the laser can be the energy, fluence, or pulse power density. The fluence is the energy per unit area, so it can have the units of Joules/cm². As an example, in dermatological applications or applications through the skin it may be advantageous to use fluences less than approximately 250 J/cm² to avoid burning or charring the epidermis layer. For example, therapeutic procedures may benefit from having fluences in the range of approximately 30 to 250 J/cm², preferably in the range of 50 to 200 J/cm². In another embodiment, it may even be advantageous to use lower fluence levels for therapeutic procedures to impart less pain to patients, for example in the range of approximately 30 J/cm² or less. These types of fluence levels may typically correspond to time averaged powers from the laser exceeding approximately 10 W, preferably in the power range of 10 W to 30 W, but perhaps as high as 50 W or more. Although particular fluence and power ranges are provided by way of example, other powers and fluences can be used consistent with this disclosure.

Although the output from a fiber laser may be from a single or multi-mode fiber, different spatial spot sizes or spatial profiles may be beneficial for different applications. For example, in some instances it may be desirable to have a series of spots or a fractionated beam with a grid of spots. In one embodiment, a bundle of fibers or a light pipe with a plurality of guiding cores may be used. In another embodiment, one or more fiber cores may be followed by a lenslet array to create a plurality of collimated or focused beams. In yet another embodiment, a delivery light pipe may be followed by a grid-like structure to divide up the beam into a plurality of spots. These are specific examples of beam shaping, and other apparatuses and methods may also be used and are consistent with this disclosure.

Also, various types of damage mechanisms are possible in biological tissue. In one embodiment, the damage may be due to multi-photon absorption, in which case the damage can be proportional to the intensity or peak power of the laser. For this embodiment, lasers that produce short pulses with high intensity may be desirable, such as the output from mode-locked lasers. Alternative laser approaches also exist, such as Q-switched lasers, cavity dumped lasers, and active or passive mode-locking. In another embodiment, the damage may be related to the optical absorption in the material. For this embodiment, the damage may be proportional to the fluence or energy of the pulses, perhaps also the time-averaged power from the laser. For this example, continuous wave, pulsed, or externally modulated lasers may be used, such as those exemplified in FIGS. 19-25. In one embodiment, laser pulses that are longer than approximately 100 nanoseconds to as long as 10 seconds or longer may be employed.

Particularly in the example when the damage may be related to the optical absorption, it may be beneficial to also consider the thermal diffusion into the surrounding tissue. As an example, the thermal diffusion time into tissue may be in the millisecond to second time range. Therefore, for pulses shorter than about several milliseconds, the heat may be generated locally and the temperature rise can be calculated based on the energy deposited. On the other hand, when longer pulses that may be several seconds long are used, there can be adequate time for thermal diffusion into the surrounding tissue. In this example, the diffusion into the surrounding tissue should be considered to properly calculate the temperature rise in the tissue. For these longer pulses, the particular spot exposed to laser energy will reach closer to thermal equilibrium with its surroundings. Moreover, another adjustable parameter for the laser pulses may be the rise and fall times of the pulses. However, these may be less important when longer pulses are used and the damage is related to the energy or fluence of the pulses.

Beyond having a pulse width, the laser output can also have a preferred repetition rate. For pulse repetition rates above around 10 MHz, where multiple pulses fall within a thermal diffusion time, the tissue response may be more related to the energy deposited or the fluence of the laser beam. The separation between pulses or a sub-group of pulses may also be selected so that the tissue sample can reach thermal equilibrium between pulses. Also, the pulse pattern may or may not be periodic. In one embodiment, there may be several pulses used per spot, where the pulse pattern is selected to obtain a desired thermal profile. The laser beam may then be moved to a new spot and then another pulse train delivered to that spot. In one embodiment, there can be several seconds of pre-cooling, the laser can be exposed on the tissue for several seconds, and then there may also be post-cooling. Although particular examples of laser duration and repetition rate are described, other values may also be used consistent with this disclosure. For example, depending on the application and mechanisms, the pulse rate could range all the way from continuous wave to 100's of Megahertz.

Described herein are just some examples of the beneficial use of infrared laser treatment based on using focused light and/or surface cooling. However, many other medical procedures can use the infrared light consistent with this disclosure and are intended to be covered by the disclosure. For example, although non-invasive vasectomy has been described in detail in various representative embodiments, more generally the focused infrared light may be used to thermally coagulate or occlude relatively shallow vessels non-invasively or minimally invasively while preserving or minimizing damage to the top layer of the skin or tissue. Other applications where this more general technique may be beneficial include treatment of varicose veins, treatment of hemorrhoids, or perhaps treatment of finger or toe nails from fungal infection.

Although the present disclosure has been described in several embodiments, a myriad of changes, variations, alterations, transformations, and modifications may be suggested to one skilled in the art, and it is intended that the present disclosure encompass such changes, variations, alterations, transformations, and modifications as falling within the spirit and scope of the appended claims.

While exemplary embodiments are described above, it is not intended that these embodiments describe all possible forms of the disclosure. Rather, the words used in the specification are words of description rather than limitation, and it is understood that various changes may be made without departing from the spirit and scope of the disclosure. Additionally, the features of various implementing embodiments may be combined to form further embodiments of the disclosure. While various embodiments may have been described as providing advantages or being preferred over other embodiments with respect to one or more desired characteristics, as one skilled in the art is aware, one or more characteristics may be compromised to achieve desired system attributes, which depend on the specific application and implementation. These attributes include, but are not limited to: cost, strength, durability, life cycle cost, marketability, appearance, packaging, size, serviceability, weight, manufacturability, ease of assembly, etc. The embodiments described herein that are described as less desirable than other embodiments or prior art implementations with respect to one or more characteristics are not outside the scope of the disclosure and may be desirable for particular applications.

What is claimed is:

1. A therapeutic system comprising:
a light source generating an output optical beam, comprising:
a plurality of semiconductor sources generating an input optical beam;
a multiplexer configured to receive at least a portion of the input optical beam and to form an intermediate optical beam; and
one or more fibers configured to receive at least a portion of the intermediate optical beam and to form the output optical beam, wherein the output optical beam comprises one or more optical wavelengths, and wherein at least a portion of the one of more fibers is a fused silica fiber with a core diameter less than approximately 400 microns;
an interface device configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample; and
wherein at least the part of the delivered portion of the output optical beam penetrates into the sample a depth of 1.5 millimeters or more, wherein at least some of the part of the delivered portion of the output optical beam is at least partially absorbed in the sample to thermally damage at least a part of the sample, and wherein the output optical beam comprises a fluence less than about 250 Joules per centimeter squared.

2. The system of claim 1, wherein the damage to at least the part of the sample is a thermal coagulation or occlusion procedure, and the sample comprises a skin.

3. The system of claim 1, wherein the output optical beam comprises a pulse width less than several milliseconds, and wherein at least a portion of the one or more optical wavelengths is near 980 nanometers.

4. The system of claim 1, wherein the one or more lenses comprise a cylindrical lens, and wherein the one or more lenses focus at least the part of the delivered portion of the output optical beam on the sample so that the focused output optical beam overcomes a Beer's law attenuation in the sample.

5. The system of claim 1, wherein the fluence of the part of the delivered portion of the output optical beam is between approximately 30 and about 250 Joules per centimeter squared.

6. A therapeutic system comprising:
a light source generating an output optical beam, comprising:
one or more semiconductor sources generating an input optical beam;
one or more fibers configured to receive at least a portion of the input optical beam and to form an intermediate optical beam; and
a light guide configured to receive at least a portion of the intermediate optical beam and to form the output optical beam, wherein the output optical beam comprises one or more optical wavelengths;
an interface device configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample, and wherein the interface device is a non-invasive device;
wherein at least some of the part of the delivered portion of the output optical beam penetrates into the sample a depth of 1.5 millimeters or more, and wherein at least some of the part of the delivered portion of the output optical beam is at least partially absorbed in the sample to thermally damage at least a part of the sample.

7. The system of claim 6, wherein the light source comprises a plurality of semiconductor sources generating the input optical beam and a multiplexer configured to receive at least a part of the input optical beam and further coupled to the one or more fibers.

8. The system of claim 6, wherein the semiconductor sources are selected from the group consisting of semiconductor lasers, super-luminescent diodes, and light emitting diodes.

9. The system of claim 6, wherein at least a portion of the one or more optical wavelengths is near 980 nanometers.

10. The system of claim 6, wherein the one or more lenses focus at least the part of the delivered portion of the output optical beam on the sample so that the focused output optical beam overcomes a Beer's law attenuation in the sample.

11. The system of claim 6, wherein the surface cooling apparatus is selected from the group consisting of a cryo-spray, an air cooling and a liquid cooled surface approximately in contact with the sample.

12. The system of claim 6, wherein the damage to at least the part of the sample is a thermal coagulation or occlusion procedure.

13. The system of claim 6, wherein the sample comprises a vas deferens and a scrotum skin.

14. The system of claim 6, wherein the output optical beam comprises a pulse width less than several milliseconds.

15. The system of claim 6, wherein the one or more lenses comprise a cylindrical lens or a lenslet array.

16. The system of claim 6, wherein the output optical beam comprises a fluence less than approximately 250 Joules per centimeter squared.

17. A method of therapy comprising:
generating an output optical beam, comprising:
generating an input optical beam from one or more semiconductor sources;
forming an intermediate optical beam after propagating at least a portion of the input optical beam through one or more fibers; and
guiding at least a portion of the intermediate optical beam and forming the output optical beam, wherein the output optical beam comprises one or more optical wavelengths;
receiving at least a received portion of the output optical beam and delivering a delivered portion of the output optical beam to a sample;
focusing at least a part of the delivered portion of the output optical beam on the sample;
cooling a top surface of the sample;
absorbing at least some of the part of the delivered portion of the output optical beam in the sample; and
damaging thermally at least a part of the sample through a thermal coagulation or occlusion procedure.

18. The method of claim 17, wherein the part of the delivered portion of the output optical beam comprises a fluence between approximately 30 and about 250 Joules per centimeter squared, and wherein the sample comprises a skin.

19. The method of claim 17, wherein the output optical beam comprises a pulse width less than several milliseconds, and wherein at least a portion of the one or more optical wavelengths is near 980 nanometers.

20. The method of claim 17, wherein at least some of the part of the delivered portion of the output optical beam penetrates into the sample a depth of 1.5 millimeters or more.
